(19) United States
(12) Patent Application Publication
(10) Pub. No.: US 2023/0250194 A1
(43) Pub. Date: Aug. 10, 2023

(54) COMPOSITIONS, SYSTEMS AND METHODS FOR STIMULATING CELLS

(71) Applicant: StemCell Technologies Canada Inc., Vancouver (CA)

(72) Inventors: Eric OUELLET, Vancouver (CA); Andy KOKAJI, Vancouver (CA)

(21) Appl. No.: 18/012,557

(22) PCT Filed: Jun. 29, 2021
(86) PCT No.: PCT/CA2021/050891

§ 371 Date: Dec. 22, 2022

Related U.S. Application Data
(60) Provisional application No. 63/045,589, filed on Jun. 29, 2020.

(51) Int. Cl. C07K 16/46; A61P 37/02

(52) U.S. Cl. CPC C07K 16/468; A61P 37/02; C07K 2317/31

(57) ABSTRACT

Provided are compositions comprising a target cell binding moiety conjugated with a nucleic acid polymer, the nucleic acid polymer characterized by a predicted melting temperature range (of secondary structures thereof) and/or a predicted free energy range (of self-hybridization). The compositions may be included in kits or systems, together with one or more complementary oligonucleotides, competitor oligonucleotides, anti-competitor oligonucleotides or the like. The compositions, kits, and systems may be used in methods of stimulating cells, quenching the stimulation of cells, modulating the stimulation of cells, or tagging (stimulated or unstimulated) cells to be analyzed.

### RELATED APPLICATION

This application claims the benefit of U.S. Provisional Patent Application No. 63/045,589, filed Jun. 29, 2020; the entire content of which is hereby incorporated by reference in its entirety.

### TECHNICAL FIELD

This disclosure relates to cell biology, and more particularly to compositions and methods for stimulating cells in culture.

### BACKGROUND

Host tolerance, innate immunity, and adaptive immunity are complex and critical components of human health. Lymphocytes are important cell types for carrying out these functions. T lymphocytes comprise various subsets including: regulatory T cells, which play a major role in the maintenance of host tolerance; and effector subsets, such as CD4+T helper cells and CD8+ cytotoxic T cells, which carry out immune responses according to environmental stimuli. B lymphocytes comprise various subsets including plasma cells and memory B cells, which produce antibodies or may be stimulated to produce antibodies. Natural killer (NK) cells are critical to the innate immune system and, among other functions, are known to provide rapid responses to infections and to respond to tumour formation.

Methods for the growth and propagation of lymphocytes in vitro have been based upon a number of different approaches. In some circumstances they may be activated and expanded by use of accessory cells and exogenous growth factors, such as antigen presenting cells and cytokines/growth factors. This requires the presence and replenishment of accessory cells and cytokines/growth factors during the course of activation and/or expansion.

Alternatively, reagents used for the activation and expansion of lymphocytes involve a combination of direct or indirect immobilization of activating antibodies, on a solid phase surface such as a plate or on a magnetic bead. In addition to the primary activation signal provided by the immobilized antibodies, a secondary co-stimulation signal provided by accessory antibodies may be required. Exogenous growth factors or cytokines such as IL-2 can also be added to enhance cell proliferation.

Antibodies against lymphocyte surface antigens are a critical component in many cell stimulation protocols. For the activation of human T lymphocytes, it was first demonstrated by Dixon et al., that immobilized anti-CD3 antibodies could mediate human T cell activation and expansion in the absence of cognate antigen recognition by the T cell receptor. Anti-CD3 initiates the activation and proliferation signaling cascade by crosslinking the components of the T cell receptor complex on the surface of T cells; thus their requirement for immobilization. It was subsequently shown by Baroja et al., that a second signal from either an immobilized or soluble anti-CD28 stimuli was required for full T cell activation in combination with immobilized anti-CD3. Additional costimulatory signals provided through adhesion ligands such as CD2, LFA-1 and other TNF family members such as CD137 (4-1BB) can provide additional proliferative or survival signals to the T cells (Smith-Garvin et. al).

For the activation of bone marrow-derived “B” lymphocytes (commonly referred to as B cells), a B cell receptor (BCR) thereof binds either soluble or membrane-bound antigens resulting in the formation of BCR microclusters that trigger downstream signaling cascades. T cell-dependent B cell activation involves the interaction of these two lymphocytes to form a stable attraction via binding CD40. It was first demonstrated by Banchereau et al. that sustained proliferation of B cells could be achieved when IL-4 and monoclonal antibodies to CD40 were presented in a cross-linked fashion on a mouse fibroblastic cell line transfected with the human Fc receptor FCγRII/CDw32. Given the important role of T cells in T cell-dependent B cell activation, various T cell-derived cytokines are often necessary to promote growth and differentiation of B cells. For example, IL-2, IL-4 and IL-5 have been demonstrated to support the maturation of primitive B cells to antibody secreting cells.

For the activation of NK cells, an array of inhibitory and activating receptors on the surface thereof are involved in the recognition of a target cell, and upon achieving a sufficient threshold of signal target cell lysis may be triggered. WO2004/056392 discloses proliferating PBMC-derived human NK cells by treatment with soluble anti NK cell receptor antibodies, such as NKp30 and NKp46. Further, U.S. Pat. No. 5,919,700 describes a role for IL-16 for inducing proliferation of NK cells. It was also shown by Bryceson et al. (2006, Blood, 107: 159-166) that in the absence of exogenous cytokines, activation of resting NK cells can be induced by combinations of activating receptors, such as NKp46 (CD335) and CD2.

In view of the important role of lymphocytes in various aspects of immunity, there is a need for low-cost, gentle and effective strategies to robustly activate such cells. Further, the ability to modulate the activation of lymphocytes in situ would permit a new way to study this phenomenon, while expanding therapeutic possibilities.

### SUMMARY OF THE DISCLOSURE

The present disclosure relates to compositions and methods for stimulating target cells in a sample of cells.

In one aspect of this disclosure are provided cell stimulation compositions, comprising a first binding moiety capable of binding a target antigen; and a first nucleic acid polymer conjugated directly or indirectly to the first binding moiety. In one embodiment, a sequence of the first nucleic acid polymer has or comprises a melting temperature of predicted secondary structure(s) about 60° C. or less.

In one embodiment, the first binding moiety is an antibody, or a fragment thereof, a peptide, an aptamer, an affimer, or a small molecule.

In one embodiment, a predicted free energy of the first nucleic acid polymer to self-hybridize at 150 mM monovalent ion concentration is between about +1 kcal/mol and −10 kcal/mol. In one embodiment, the predicted free energy of the first nucleic acid polymer at 150 mM monovalent ion concentration is negative.

In one embodiment, the melting temperature of predicted secondary structure(s) of the first nucleic acid polymer is between about 50° C. and 20° C. or less.

In one embodiment, the first nucleic acid polymer is at least 10 nucleotides.

In one embodiment, the first nucleic acid polymer or a complementarity region thereof is complementary to a second nucleic acid polymer.

In one embodiment, the second nucleic acid polymer is directly or indirectly conjugated to a second binding moiety.

In one embodiment, a bispecific complex of binding moieties is formed by hybridization of the first nucleic acid polymer and the second nucleic acid polymer.

In one embodiment, the first binding moiety and the second binding moiety bind the same target antigen. In one embodiment, the first binding moiety and the second binding moiety bind different target antigens.

In one embodiment, the first nucleic acid polymer is conjugated to the first binding moiety by nucleophilic substitution, electrophilic substitution, alkylation, oxidation, condensation, cycloaddition or Staudinger ligation reaction. In one embodiment, the second nucleic acid polymer is conjugated to the second moiety by nucleophilic substitution, electrophilic substitution, alkylation, oxidation, condensation, cycloaddition or Staudinger ligation reaction.

In one embodiment, the cell stimulation is soluble. In the same or different embodiment, the second nucleic acid polymer conjugated to the second binding moiety is soluble.

In one embodiment, the target antigen is a component of the T cell receptor complex. In one embodiment, the component of the T cell receptor complex is CD3 epsilon, CD3 delta, CD3 gamma, CD3 zeta, TCR alpha, TCR beta, TCR gamma, or TCR delta.

In another aspect of this disclosure are provided methods of stimulating target cells in a sample of cells, the methods comprising contacting the sample of cells with a first binding moiety conjugated directly or indirectly to a first nucleic acid polymer, the first binding moiety capable of binding a target antigen on the target cells, wherein the first binding moiety conjugated directly or indirectly to a first nucleic acid polymer forms a cell stimulation composition, and incubating the target cells having been bound by the first binding moiety of the cell stimulation composition.

In one embodiment, the methods further comprise contacting the sample of cells with a co-stimulatory binding moiety capable of binding a co-stimulatory antigen on the target cells, either before, contemporaneous with the cell stimulation composition, or after contacting the sample of cells with the cell stimulation composition.

In one embodiment, the first binding moiety is an antibody, or a fragment thereof, a peptide, an aptamer, an affimer, or a small molecule.

In one embodiment, a predicted free energy of the first nucleic acid polymer to self-hybridize at 150 mM monovalent ion concentration is between about +1 kcal/mol and −10 kcal/mol. In one embodiment, a predicted free energy of the first nucleic acid polymer at 150 mM monovalent ion concentration is negative.

In one embodiment, the first nucleic acid polymer comprises a melting temperature of predicted secondary structures about 60° C. or less. In one embodiment, the melting temperature of predicted secondary structures of the first nucleic acid polymer is between about 50° C. and 20° C. or less.

In one embodiment, the first nucleic acid polymer is at least 10 nucleotides.

In one embodiment, the target cells are T cells, NK cells, or B cells.

In one embodiment, the first target antigen (of the target cells) for T cells is CD3 epsilon, CD3 delta, CD3 gamma, CD3 zeta, TCR alpha, TCR beta, TCR gamma, or TCR delta.

In one embodiment, the first antigen (of the target cells) for NK cells is NKp30, NKp44, and NKp46, a C-type lectin-like receptor such as NKG2D, CD335, CD94, CD2, CD16 binding to Fc regions of antibodies, CD122, CD132, IL-15 receptor alpha.

In one embodiment, the first target antigen (of the target cells) for B cells is CD40, an anti-immunoglobulin, CD79a, or CD79b.

In one embodiment, the methods further comprise increasing proliferation of the target cells, such as after contacting the target cells with a cell stimulation complex and/or a co-stimulatory binding moiety and/or a second binding moiety.

In one embodiment, the co-stimulatory binding moiety is an antibody, or a fragment thereof, a peptide, an aptamer, an affimer, or a small molecule.

In one embodiment, the co-stimulatory antigen for T cells is CD28, CD137, CD2, CD134, CD152, CD278, CD27, or CD279.

In one embodiment, the co-stimulatory antigen for NK cells is a different one (relative to the first antigen) of NKp30, NKp44, and NKp46, a C-type lectin-like receptor such as NKG2D, CD335, CD94, CD2, CD16 binding to Fc regions of antibodies, CD122, CD132, IL-15 receptor alpha.

In one embodiment, the co-stimulatory antigen for B cells is a different one (relative to the first antigen) of CD40, an anti-immunoglobulin, CD79a, or CD79b.

In one embodiment, the methods further hybridizing a second nucleic acid polymer to the first nucleic acid polymer or a complementarity region thereof. In one embodiment, a complementarity of the second nucleic acid polymer to the first nucleic acid polymer or the complementary region thereof is between about 40% and 100%.

In one embodiment, hybridizing the second nucleic acid polymer to the first nucleic acid polymer, or the complementarity region thereof, is either before, contemporaneous with, or after contacting the sample of cells with the second binding moiety quenches or modulates a level of target cell stimulation.

In one embodiment, the methods further comprise displacing the second nucleic acid polymer from the first nucleic acid polymer, or the complementarity region thereof, by competition with a competitor nucleic acid polymer, the competitor nucleic acid polymer having a higher degree of complementarity for the second nucleic acid polymer than the second nucleic acid polymer for the first nucleic acid polymer or the complementarity region thereof.

In one embodiment, the second nucleic acid polymer is directly or indirectly conjugated to a second binding moiety. In one embodiment, the second binding moiety is the same as the first binding moiety.

In one embodiment, the methods further comprise forming a bispecific complex of binding moieties by virtue of interaction (e.g. hybridization) between the first nucleic acid polymer and the second nucleic acid polymer.

In one embodiment, the cell stimulation composition is soluble. In the same or a different embodiment, the second nucleic acid polymer conjugated to the second binding moiety is soluble.

In another aspect of this disclosure are provided methods of tagging a target cell in a sample of cells to be analyzed for a biomolecular signature, the methods comprising contacting the target cell with a first binding moiety conjugated directly or indirectly to a first nucleic acid polymer, the first binding moiety capable of binding a target antigen on the target cell, wherein the first binding moiety conjugated directly or indirectly to a first nucleic acid polymer forms a cell stimulation composition; and inhibiting stimulation of the target cell bound by the cell stimulation composition by hybridizing a second nucleic acid polymer to the first nucleic acid polymer, or a complementarity region thereof.

In one embodiment, hybridizing the second nucleic acid polymer is before or contemporaneous with contacting the target cell with the cell stimulation composition.

In one embodiment, the first binding moiety is an antibody, or a fragment thereof, a peptide, an aptamer, an affimer, or a small molecule.

In one embodiment, a predicted free energy of the first nucleic acid polymer to self-hybridize at 150 mM monovalent ion concentration is between about +1 kcal/mol and −10 kcal/mol. In one embodiment, a predicted free energy of the first nucleic acid polymer at 150 mM monovalent ion concentration is negative.

In one embodiment, the first nucleic acid polymer comprises a melting temperature of predicted secondary structures about 60° C. or less. In one embodiment, the melting temperature of predicted secondary structures of the first nucleic acid polymer is between about 50° C. and 20° C. or less.

In one embodiment, the first nucleic acid polymer is at least 10 nucleotides.

In one embodiment, the target cells are T cells, NK cells, or B cells.

In one embodiment, the first antigen (on the target cells) for T cells is CD3 epsilon, CD3 delta, CD3 gamma, CD3 zeta, TCR alpha, TCR beta, TCR gamma, or TCR delta.

In one embodiment, the first antigen (on the target cells) for NK cells is NKp30, NKp44, and NKp46, a C-type lectin-like receptor such as NKG2D, CD335, CD94, CD2, CD16 binding to Fc regions of antibodies, CD122, CD132, IL-15 receptor alpha.

In one embodiment, the first antigen (on the target cells) for B cells is CD40, an anti-immunoglobulin, CD79a, or CD79b.

In one embodiment, the cell stimulation composition is soluble.

In another aspect of this disclosure are provided kits comprising a cell stimulation composition as described herein. In one embodiment, kits of this disclosure further comprise any other the component described herein, such as one or more of a second nucleic acid polymer (whether or not conjugated to a second binding moiety), a co-stimulatory binding moiety, a competitor oligonucleotide, an anti-competitor oligonucleotide (to compete for the competitor oligonucleotide); and buffers for reconstituting any one or more of the foregoing. In one embodiment, the kits further comprise culture media appropriate for culturing the sample of cells and/or the target cell(s) included within the sample of cells.

Other features and advantages of the presently disclosed subject-matter will become apparent from the following detailed description. It should be understood, however, that the detailed description and the specific examples while indicating preferred embodiments of the disclosed subject-matter are given by way of illustration only, since various changes and modifications within the spirit and scope of the invention will become apparent to those skilled in the art from this detailed description.

### BRIEF DESCRIPTION OF THE DRAWINGS

FIG. 1 shows illustrations of various embodiments of cell stimulation compositions or systems including cell stimulation compositions. In one embodiment, a binding moiety “B”, specific for a target antigen “A”, is conjugated either directly or indirectly to a first nucleic acid polymer “C” (A). The first nucleic acid polymer, or a complementarity region thereof, of the cell stimulation composition of (A) may hybridize to a second nucleic acid polymer (or, in some embodiments a competitor nucleic acid polymer) “C” (B). In one embodiment, the second nucleic acid polymer may be directly or indirectly conjugated to a second binding moiety “D”, specific for a target antigen “E” (C). In some embodiments the first binding moiety “B” and the second binding moiety “D” bind the same target antigen, and in other embodiments they bind different target antigens.

FIG. 2 shows representative flow cytometry plots of T cells isolated by negative selection. T cells were analyzed via flow cytometry from a viable CD45+ gate for their pre-enrichment (A) and post-enrichment (B) purity (via their CD3 marker). Additionally flow analysis of the various T cells subsets expressing either CD4 or CD8, or both (C) confirms their retention following isolation (D).

FIG. 3 shows flow cytometry histograms of competition assays using APC-conjugated anti-CD3 antibodies and different concentrations (0 μg/mL, 0.1 μg/mL, 0.5 μg/mL, and 5 μg/mL) of a soluble cell stimulation composition of this disclosure binding to cells in a sample of peripheral blood mononuclear cells. Panel (A) shows histograms using anti-human CD3 IgG monoclonal antibodies not conjugated to nucleic acid polymer, and panel (B) shows histograms using anti-human CD3 IgG monoclonal antibodies conjugated to nucleic acid polymers (i.e. cell stimulation compositions).

FIG. 4 shows a bar chart of T cell activation. CD25 expression level was determined in different T cell subsets at day 3 following co-stimulation with different concentrations of a soluble cell stimulation composition and soluble anti-CD28 IgG monoclonal antibodies. Unstimulated cells (no antibody treatment), unconjugated anti-CD3 IgG monoclonal antibodies, and Dynabeads™ were included as controls. Data represent mean±SD frequency of CD25⁺ cells, n=2.

FIG. 5 shows a bar chart of T cell activation. CD25 expression level was determined in different T cell subsets on day 2 (A) or day 3 (B) following co-stimulation with a soluble cell stimulation composition or control compositions, and soluble anti-CD28 IgG monoclonal antibodies. Unstimulated cells (no antibody treatment), unconjugated anti-CD3 IgG monoclonal antibodies, free nucleic acid polymers either alone or together with unconjugated anti-CD3 IgG, and Dynabeads™ were included as controls. Data represent mean±SD frequency of CD25⁺ cells, n=1.

FIG. 6 shows a bar chart of T cell activation. CD25 expression level was determined in different T cell subsets on day 3-following co-stimulation using soluble cell stimulation compositions comprising different antibodies (as indicated) and soluble anti-human CD28 IgG monoclonal antibodies. Unstimulated cells (no antibody treatment), unconjugated anti-CD3 IgG monoclonal antibodies, and Dynabeads™ were included as controls. Data represent mean±SD frequency of CD25⁺ cells, n=1.

FIG. 7 shows a bar chart of quenched T cell activation. CD25 expression level was determined in different T cell subsets on day 3. The activation of T cells co-stimulated with the indicated cell stimulation composition and anti-CD28 IgG monoclonal antibodies could be quenched by pre-hybridizing a nucleic acid polymer complementary to at least a portion of the nucleic acid polymer of the cell stimulation composition. Unconjugated anti-CD3 IgG monoclonal antibodies and Dynabeads™ were included as controls. Data represent mean±SD frequency of CD25⁺ cells, n=1.

FIG. 8 shows a bar chart of modulated T cell activation. CD25 expression level was determined in different T cell subsets on day 3. The response of in-culture T cells co-stimulated with the indicated cell stimulation composition and anti-CD28 IgG monoclonal antibodies could be modulated by hybridizing a nucleic acid polymer complementary to at least a portion of the nucleic acid polymer of the cell stimulation composition in situ. Unconjugated anti-CD3 IgG monoclonal antibodies and Dynabeads™ were included as controls. Data represent mean±SD frequency of CD25⁺ cells, n=1.

FIG. 9 shows a bar chart of T cell activation. CD25 expression level was determined in different T cell subsets on day 3 following co-stimulation using different soluble cell stimulation compositions (as indicated) and soluble anti-CD28 IgG monoclonal antibodies. (A) The tested cell stimulation compositions include variations of oligonucleotide A having been engineered with point mutations to modify the melting temperature of its predicted secondary structures. Unstimulated cells (no antibody treatment), unconjugated anti-CD3 IgG monoclonal antibodies, and Dynabeads™ were included as controls. Data represent mean±SD frequency of CD25⁺ cells, n=1. Optical micrographs of activated T cells taken at 72 hours following initial stimulation are also included for the tested cell stimulation composition variants, A (B), A_var1 (C) and A_var2 (D).

FIG. 10 shows a bar chart of T cell activation. CD25 expression level was determined in different T cell subsets on day 3 following co-stimulation using different soluble cell stimulation compositions (as indicated) and soluble anti-CD28 IgG monoclonal antibodies. (A) The different cell stimulation compositions include variations of oligonucleotide B having been engineered with point mutations to modify the melting temperature of its predicted secondary structures. Unstimulated cells (no antibody treatment), unconjugated anti-CD3 IgG monoclonal antibodies, and Dynabeads™ were included as controls. Data represent mean±SD frequency of CD25⁺ cells, n=2. Optical micrographs of activated T cells taken at 72 hours following initial stimulation are also included for the tested cell stimulation composition variants, B (B), B_var1 (C) and B_var2 (D).

FIG. 11 shows a bar chart of T cell activation. CD25 expression level was determined in different T cells subsets on day 3 following co-stimulation using different soluble cell stimulation compositions (as indicated) and soluble anti-CD28 IgG monoclonal antibodies. (A) The different cell stimulation compositions include variations of oligonucleotide C having been engineered with point mutations to modify the melting temperature of its predicted secondary structures. Unstimulated cells (no antibody treatment), unconjugated anti-CD3 IgG monoclonal antibodies, and Dynabeads™ were included as controls. Data represent mean±SD frequency of CD25⁺ cells, n=2. Optical micrographs of activated T cells taken at 72 hours following initial stimulation are also included for the tested cell stimulation composition variants, C (B), C_var1 (C) and C_var2 (D).

FIG. 12 shows the reduction of T cell activation using different oligonucleotides having complementarity to different regions of a cell stimulation composition. The bar chart in panel A summarizes CD25 expression level in different T cell subsets after treatment with a cell stimulation composition (CD3-oligoC) and anti-CD28 IgG monoclonal antibodies. CD3-oligoC was separately pre-hybridized with 5 different complementary oligonucleotides (CC-1, CC-3, CC-12, CC-23, and both CC-1 and CC-3) and the level of T cell activation was assessed on day 3. Unconjugated anti-CD3 IgG monoclonal antibodies and Dynabeads™ were included as controls. Data represent mean±SD frequency of CD25⁺ cells, n=1. The bar chart in panel B summarizes the diameter of the T cells after having been treated essentially as described above, except CD3-OligoC was substituted with two other cell stimulation compositions: C_var1 and C_var2 (both complexed to an anti-human CD3 IgG). An additional control of anti-human CD3 IgG (not conjugated to an oligonucleotide) was also used.

FIG. 13 shows the results of T cell activation using different cell stimulation compositions, and the effects of a complementary oligonucleotide on quenching T cell activation. The bar chart in panel A summarizes CD25 expression level in different T cell subsets on day 3 after treatment with different cell stimulation compositions (C_var2, C2×, C3×, and C4×) and anti-CD28 IgG monoclonal antibodies. Untreated cells and unconjugated anti-CD3 IgG monoclonal antibodies were used as negative controls, and C_var2 and Dynabeads™ were included as positive controls. The bar chart in panel B summarizes the effects of different concentrations of an oligonucleotide (CC-3) having complementarity against a cell stimulation composition (anti-human CD3 IgG conjugated to oligo C4×) on quenching T cell activation. Untreated cells and unconjugated anti-CD3 IgG monoclonal antibodies were used as negative controls. Data represent mean±SD frequency of CD25⁺ cells, n=1.

FIG. 14 shows a bar chart of T cell activation. CD25 expression level was determined in different T cell subsets on day 3. T cells were co-stimulated using the soluble cell stimulation compositions (CD3-oligoD and CD3-oligoDC alone or in combination) and soluble anti-CD28 IgG monoclonal antibodies. Both CD3-oligoD and CD3-oligoDC comprised the same anti-human CD3 IgG, but differed by conjugation with each respective member of a complementary pair of oligonucleotides. Unstimulated cells and unconjugated anti-CD3 IgG monoclonal antibodies were included as controls. Data represent mean±SD frequency of CD25⁺ cells, n=2.

FIG. 15 shows a graph summarizing the activation of mouse T cells at either day 2 or day 3 following treatment with a cell stimulation composition of this disclosure. Mouse T cells were contacted with an anti-mouse CD3 IgG conjugated to either C4× or C-var2 nucleic acid polymers and anti-mouse CD28 IgG. T cell activation levels of the foregoing cell stimulation compositions was compared to T cell activation using: unconjugated, soluble anti-mouse CD3 IgG; unconjugated, plate-bound anti-mouse CD3 IgG; and Dynabeads (1:2 bead to cell ratio). Untreated cells were included as a negative control. Data represent mean±SD frequency of CD25⁺ cells, n=1.

### DETAILED DESCRIPTION

The description that follows relates to compositions and methods for stimulating target cells in a sample of cells.

Where used herein the term “cell stimulation composition” refers to a means that elicits a stimulatory response of a target cell when placed into proximity and/or contact with the cell. Depending on the nature of the cell stimulation composition and the type of target cell, the stimulatory response may include one or more of activation, proliferation, cytokine or chemokine secretion, antibody production, or target cell killing. It may be optional or even necessary in some cases to additionally use one or more co-stimulatory factors, which co-stimulatory factors may be utilized contemporaneous with or in a sequence with the cell stimulation composition, in order to elicit the stimulatory response or to enhance the stimulatory response. In a preferred embodiment, a cell stimulation composition of this disclosure is soluble. In a further preferred embodiment, a cell stimulation composition of this disclosure includes at least one of both a binding moiety and a nucleic acid polymer. In one embodiment, a cell stimulation composition of this disclosure includes one binding moiety and one nucleic acid polymer. In one embodiment, a cell stimulation composition of this disclosure includes either one binding moiety and more than one nucleic acid polymer, or more than one binding moiety and one nucleic acid polymer.

Where used herein the term “binding moiety” refers to a biological or chemical composition having affinity and specificity for one or more targets, such as a target antigen. In a preferred embodiment, the biological or chemical composition is specific for a single target or a class of related targets. The biological or chemical composition may reversibly or irreversibly bind the target(s). Binding moiety examples may include an antibody, or a fragment thereof, a peptide, an aptamer, an affimer, or a small molecule. In one embodiment, the binding moiety is an antibody, or a fragment thereof.

Where used herein the term “target antigen” refers to an antigen, or epitope thereof, present within a cell or presented on the surface of a cell, such as a target cell. In the context of this disclosure, the binding of one or more target antigens by a cell stimulation complex (along with the necessary or optional binding of different target antigens by co-stimulatory factors) elicits the stimulatory response of the target cells.

Where used herein the term “nucleic acid polymer” refers to a single stranded polymer of building block monomers. In one embodiment, the polymer consists of a single class of monomers, such as deoxynucleotides or deoxyribonucleotides. In one embodiment, the polymer consists of more than one class of monomers, such as both deoxynucleotides and deoxyribonucleotides. In one embodiment, the polymer may be a locked nucleic acid, or a peptide nucleic acid. In a preferred embodiment, the nucleic acid polymer consists of deoxynucleotides. In some embodiments, the nucleic acid polymer may include modified variants of monomers. In one embodiment, the nucleic acid polymer is an oligonucleotide.

### Compositions

In one aspect of this disclosure are described cell stimulation compositions which may be used with or added to a sample of cells. The cell stimulation compositions of this disclosure may also be used in methods of stimulating target cells, as described below in further detail.

The cell stimulation compositions of this disclosure will comprise a first binding moiety capable of binding a target antigen and a first nucleic acid polymer conjugated directly or indirectly to the first binding moiety.

The cell stimulation compositions of this disclosure should also be soluble, although this is not an absolute requirement as cell stimulation compositions immobilized on a surface may also be operative. More particularly, if the cell stimulation compositions are soluble, they should be soluble in the liquid used to bathe a sample of cells that includes target cells.

The first binding moiety may be any type of biological or chemical composition capable of binding a target. Examples of first binding moieties may include an antibody, or a fragment thereof, a peptide, an aptamer, an affimer, or a small molecule.

In one embodiment, the first binding moiety is an antibody, or an antibody fragment. The antibody, or fragment thereof, may be either monoclonal or polyclonal, but in many cases it is preferred that the antibody or fragment thereof is monoclonal.

A desirable affinity of the first binding moiety for its target antigen may be considered as binding with an equilibrium dissociation constant (KD) of less than 100 nM. Preferably the affinity of the first binding moiety for its target antigen is below 10 nM. In other cases, a weaker binding affinity for the target antigen may be desirable for individual subunits of the first binding moiety, that collectively provide the first binding moiety with high affinity binding, on the order of the foregoing. Furthermore, binding between the target antigen and first binding moiety should be highly specific.

The target antigen may be any antigen, or epitope thereof, present within a target cell or presented on the surface of a target cell. Among the most important features of the target antigen is that it is accessible and presents limited steric hindrance for binding by a cell stimulation composition, and more particularly, a first binding moiety thereof. In one embodiment, the target antigen or epitope thereof is comprised in an entity, or in an entity of a complex of entities, presented on an external surface of a cell, such as a protein, peptide, or glycoprotein, for example.

In one embodiment, the target antigen may be a component of the T cell receptor complex. For example, the component of the T cell receptor complex may be one of CD3 epsilon, CD3 delta, CD3 gamma, CD3 zeta, TCR alpha, TCR beta, TCR gamma, or TCR delta. Thus, binding of the component of the T cell receptor complex (and subsequent close association of multiple of the components) by the cell stimulation compositions of this disclosure may elicit a stimulatory response by the T cells or by different T cell subsets. In one embodiment, the stimulatory response of the T cells, or subsets thereof, may be an activation of the target T cells which may lead to a proliferation of such T cells or T cell subsets, and/or a secretion of cytokines or chemokines by such T cells or T cell subsets. In addition, the stimulation of one T cell subset, such as CD4⁺ T cells may in turn result in the stimulation of a different T cell subset, such as CD8⁺ T cells.

In one embodiment, the target antigen may be a component presented by B cells. For example, the component presented by B cells may include one of CD40, an anti-immunoglobulin, CD79a, or CD79b. Thus, binding of the component of the B cell receptor complex (and subsequent close association of multiple of the components) by the cell stimulation compositions of this disclosure may elicit a stimulatory response by the B cells or by different B cell subsets, such as an activation of the target B cells which may lead to a proliferation of such B cells or B cell subsets, and/or a production of antibodies by such B cells or B cell subsets. In addition, the stimulation of one B cell subset may in turn result in the stimulation of a different B cell subset.

In one embodiment, the target antigen may be a component presented by NK cells. For example, the component presented by NK cells may include one of NKp30, NKp44, and NKp46, a C-type lectin-like receptor such as NKG2D, CD335, CD94, CD2, CD16 binding to Fc regions of antibodies, CD122, CD132, IL-15 receptor alpha. Thus, binding of the component presented by the NK cells (and subsequent close association of multiple of the components) by the cell stimulation compositions of this disclosure may elicit a stimulatory response by the NK cells or by different NK cell subsets, such as an activation of the target NK cells which may lead to a proliferation of such NK cells or NK cell subsets, and/or a killing of target cells by such NK cells or NK cell subsets. In addition, the stimulation of one NK cell subset may in turn result in the stimulation of a different NK cell subset.

The inventors have unexpectedly discovered that cell stimulation compositions, comprising a first nucleic acid polymer conjugated directly or indirectly to a first binding moiety, may be used to stimulate target cells without the need to pre-complex two of such cell stimulation compositions to one another before coming into contact with target cells. In fact, an important feature of the first nucleic acid polymer appears to be the inability of, or a limited ability of, the first nucleic acid polymer to fold upon itself (i.e. self-hybridize) with appreciable levels of stability under typical cell culture conditions (e.g. appropriate medium and/or incubation conditions).

A first binding moiety may be conjugated to the first nucleic acid polymer using any known reaction, provided that the constitution of these components is not negatively impacted during the conjugation reaction(s). In one embodiment, the first nucleic acid polymer may be conjugated to the first binding moiety by one of nucleophilic substitution, electrophilic substitution, alkylation, oxidation, condensation, cycloaddition or Staudinger ligation reaction.

In one embodiment, the first nucleic acid polymer may be conjugated to the first binding moiety by one or more covalent bond linkages involving hydrazone, oxime, imides, sulfones, triazine, or other covalent attachment via functional groups selected from the group comprising sulfhydryl, amine, carboxyl, hydroxyl, hydrazide, aldehyde or glycans.

In one embodiment, the first nucleic acid polymer may be indirectly conjugated to the first binding moiety. In one embodiment, the indirect conjugation may be accomplished via interaction between components having been modified by biotin and avidin/streptavidin, or any similar type of mating pair.

Regardless of the specific conjugation approach taken, the person skilled in the art will readily know or know where to find the precise protocol to perform the conjugation reaction.

Since the cell stimulation compositions of this disclosure were surprisingly shown to stimulate target cells without the need to pre-complex two of such cell stimulation compositions to one another, it may be important to modify a sequence of the first nucleic acid polymer such that a melting temperature of predicted secondary structures thereof is about 60° C. or less. In one embodiment, the first nucleic acid polymer comprises a sequence wherein a melting temperature of predicted secondary structures is between about 50° C. and 10° C., or less. In one embodiment, the engineered, or otherwise, first nucleic acid polymer includes a sequence wherein melting temperature of predicted secondary structures is between about 40° C. and 20° C.

A not necessarily mutually exclusive feature of the first nucleic acid polymer may relate to its free energy. In one embodiment, a predicted free energy of the first nucleic acid polymer at 150 mM monovalent ion concentration is between about +1 kcal/mol and −10 kcal/mol. In one embodiment, a predicted free energy of the first nucleic acid polymer at 150 mM monovalent ion concentration is negative.

Depending on the application of use of the cell stimulation compositions, it may be desirable that a sequence of the first nucleic acid polymer is devoid of motifs known to trigger cellular responses, such as stimulatory responses. For example, a first nucleic acid polymer rich in CpG motifs may elicit a pro-inflammatory response via toll-like receptor (TLR) 9 recognition. Alternatively, double-stranded DNA polymers with unpaired ends (Y-form) containing guanosines may also be recognized via the cytosolic immune-sensing receptor cGAS or via AIM-1 in a sequence-independent fashion for fragments ranging between 50 to 80 bp. Furthermore, tri-phopshorylated (5′ppp) double-stranded ribonucleic acid (RNA) polymers can also elicit immune responses via RIG-1 or via TLR3 when longer than 40 bp. Those containing polyU or G/U-rich sequences may also be recognized by TLR7, for example. Single-stranded RNA polymers containing polyU/UC motifs may also elicit immune response via RIG-1 signaling.

Another important feature of the first nucleic acid polymer may be a length thereof. Since the possibility of secondary structures usually increases with the length of a single stranded first nucleic acid polymer, in most embodiments the first nucleic acid polymer is about 100 monomers long, or less. In one embodiment, the first nucleic acid polymer is about 90 monomers long, or less. In one embodiment, the first nucleic acid polymer is about 80 monomers long, or less. In one embodiment, the first nucleic acid polymer is about 70 monomers long, or less. In one embodiment, the first nucleic acid polymer is about 60 monomers long, or less. In one embodiment, the first nucleic acid polymer is about 50 monomers long, or less. In one embodiment, the first nucleic acid polymer is about 40 monomers long, or less. In one embodiment, the first nucleic acid polymer is about 30 monomers long, or less. In one embodiment, the first nucleic acid polymer is about 20 monomers long, or less. In one embodiment, the first nucleic acid polymer is at least about 10 monomers long. In one embodiment, the first nucleic acid polymer is at least about 5 monomers long.

In one embodiment, the first nucleic acid polymer (or a complementarity region thereof) is complementary to a second nucleic acid polymer. The second nucleic acid polymer may either be directly or indirectly conjugated to at least one second binding moiety, or not conjugated to at least one second binding moiety.

Similar to the first binding moiety, the second binding moiety may be an antibody, or a fragment thereof, a peptide, an aptamer, an affimer, or a small molecule. Likewise to the first binding moiety, the second binding moiety may either be soluble or immobilized on a surface. In one embodiment, the second binding moiety is soluble. In one embodiment, both the cell stimulation composition and the second binding moiety are soluble.

In embodiments where the second nucleic acid polymer is not conjugated to at least one second binding moiety, activity of the cell stimulation composition may be quenched when the first nucleic acid polymer thereof is hybridized with the second nucleic acid polymer. In such embodiments, the timing of stimulation may be controlled. Or, compositions similar to, or the same as, the cell stimulation composition of this disclosure may be used in certain next-generation sequencing approaches, and the unwitting or undesired stimulation of cells targeted for sequencing can either be prevented or limited.

In one embodiment, the second nucleic acid polymer is pre-hybridized to the first nucleic acid polymer (or the complementarity region thereof) prior to the cell stimulation composition coming into contact with the sample of cells (and the target cells therein). In such an embodiment, the stimulatory response of the target cells to the cell stimulation composition may be initially quenched. Such an “inactive” cell stimulation composition may become “activated” at an appropriate time by a competitor nucleic acid polymer, wherein a hybridization preference of the second nucleic acid polymer for the competitor nucleic acid polymer is higher than for the first nucleic acid polymer.

In one embodiment, the second nucleic acid polymer is hybridized to the first nucleic acid polymer (or a complementarity region thereof) contemporaneous with or after the cell stimulation composition comes into contact with the sample of cells (and the target cells therein). In such an embodiment, the cell stimulation composition may initially elicit the stimulatory response of the target cells. At an appropriate time the stimulatory response of the target cells to the cell stimulation composition may be quenched with a second nucleic acid polymer. At a later appropriate time, the stimulatory response may be reactivated with a competitor nucleic acid polymer, wherein a hybridization preference of the second nucleic acid polymer for the competitor nucleic acid polymer is higher than for the first nucleic acid polymer.

In embodiments where the second nucleic acid polymer is conjugated to at least one second binding moiety, the conjugation may be performed in any way known to or accessible by the person skilled in the art. For example, a second binding moiety may be conjugated to the second nucleic acid polymer using any known reaction, provided that the constitution of these components is not negatively impacted during the conjugation reaction(s). In one embodiment, the second nucleic acid polymer may be conjugated to the second binding moiety by one of nucleophilic substitution, electrophilic substitution, alkylation, oxidation, condensation, cycloaddition or Staudinger ligation reaction.

In one embodiment, the second nucleic acid polymer may be conjugated to the second binding moiety by one or more covalent bond linkages involving hydrazone, oxime, imides, sulfones, triazine, or other covalent attachment via functional groups selected from the group comprising sulfhydryl, amine, carboxyl, hydroxyl, hydrazide, aldehyde or glycans.

In one embodiment, the second nucleic acid polymer may be indirectly conjugated to the second binding moiety. In one embodiment, the indirect conjugation may be accomplished via interaction between components having been modified by biotin and avidin/streptavidin, or any similar type of mating pair.

In one embodiment, a cell stimulation composition may be comprised in a bispecific complex of binding moieties. A bispecific complex of binding moieties may be formed by virtue of hybridizing the first nucleic acid polymer and the second nucleic acid polymer (each being directly or indirectly conjugated respectively to first and second binding moieties). In one embodiment, the first binding moiety and the (soluble) second binding moiety bind the same target antigen. In one embodiment, the first binding moiety and the (soluble) second binding moiety bind different target antigens. In one embodiment, the target antigens (respectively bound by the first binding moiety and the second binding moiety) are on the same target cell type. In one embodiment, the target antigens (respectively bound by the first binding moiety and the second binding moiety) are on different target cell types.

In one embodiment, the second nucleic acid polymer is pre-hybridized to the first nucleic acid polymer (or complementarity region thereof) prior to the bispecific complex of binding moieties coming into contact with the sample of cells (and the target cells therein). In such an embodiment, the bispecific complex of binding moieties may initially elicit the stimulatory response of the target cells. At an appropriate time, the stimulatory response of the target cells to the bispecific complex of binding moieties may be quenched with one or more competitor nucleic acid polymer, wherein a hybridization preference of the competitor nucleic acid polymer(s) for the first nucleic acid polymer and/or the second nucleic acid polymer is higher than a hybridization preference of the first nucleic acid polymer for the second nucleic acid polymer. At a later appropriate time the stimulatory response may be reactivated with an anti-competitor nucleic acid polymer, wherein a hybridization preference of the anti-competitor nucleic acid polymer for the competitor nucleic acid polymer is higher than a hybridization preference of the competitor nucleic acid polymer for the first nucleic acid polymer or the second nucleic acid polymer.

In one embodiment, formation of the bispecific complex of binding moieties may initially be perturbed by hybridization of a competitor nucleic acid polymer to either or both the first nucleic acid polymer and/or second nucleic acid polymer, wherein a hybridization preference of the competitor nucleic acid polymer(s) for the first nucleic acid polymer and/or the second nucleic acid polymer is higher than a hybridization preference of the first nucleic acid polymer for the second nucleic acid polymer. Thus, the stimulatory response of the target cells may initially be quenched. Such an “inactive” bispecific complex of binding moieties may become “active” at an appropriate time with one or more anti-competitor nucleic acid polymer(s), wherein a hybridization preference of the anti-competitor nucleic acid polymer(s) for the competitor nucleic acid polymer(s) is higher than a hybridization preference of the competitor nucleic acid polymer(s) for the first nucleic acid polymer and/or the second nucleic acid polymer.

If there is a concern in applications of use that the uncomplexed components of the bispecific complex of binding moieties may individually trigger the stimulatory response in the target cells, this may be controlled in accordance with the teachings herein. For example, a sequence of the first and/or second nucleic acid polymers may be modified or engineered to have disabling secondary structures (i.e. having melting temperatures above 50° C. or 60° C., or higher). By way of additional example, the uncomplexed components of the bispecific complex of binding moieties may be “neutralized” or “disabled” with appropriate neutralizing or disabling nucleic acid polymers that hybridize to the first and second nucleic acid polymers.

Accordingly, although the cell stimulation compositions of this disclosure were surprisingly shown to stimulate target cells without the need to pre-complex two of such cell stimulation compositions to one another, it may nevertheless be desirable in some cases.

Thus, in addition to compositions, this disclosure also contemplates systems comprising cell stimulation compositions and second nucleic acid polymers. In some embodiments, the systems may further comprise competitor nucleic acid polymers. In some embodiments, the systems may further comprise anti-competitor nucleic acid polymers. In some embodiments, the systems may further comprise neutralizing or disabling nucleic acid polymers. In some embodiments, the components of such systems may be packaged in a kit or kits.

### Methods

In another aspect of this disclosure are described methods of stimulating target cells in a sample of cells, using such cell stimulation compositions as have been described in the foregoing. Alternatively, the compositions of this disclosure may be used in methods of specifically binding or tagging target cells (for downstream assays of interested), either without unwittingly triggering stimulation of the target cells or specifically intending to trigger stimulation of the target cells. For example, such compositions may be used in proteo-transcriptomic analyses, however it may be either desirable or undesiderable to assess the transcriptome of the target call in either the activated or unactivated (i.e. stimulated vs unstimulated) state. In such an application, it will be clear how to carry out an appropriate method based on the description(s) provided above in respect of the compositions of this disclosure. Briefly, such a method may comprise tagging or binding target cell(s) with a composition of this disclosure and obtaining a biomolecular (e.g. transcriptome) signature of the target cell(s). A composition used in such a method may be hybridized with a second nucleic acid polymer before, as, or after the composition binds the target cell(s). In some embodiments, it may be desirable to compare the transcriptome signature of a population of target cells stimulated with a composition of this disclosure to a population of target cells (bound by) but not stimulated with a composition of this disclosure.

The target cell(s) may be any type of cell of any species, provided it presents a target antigen and the binding of such target antigen by a cell stimulation composition of this disclosure effects a stimulatory response of the cell.

The target antigen may be any antigen, or epitope thereof, present within a target cell or presented on the surface of a target cell. Among the most important features of the target antigen is that it is accessible to and presents limited steric hindrance for binding by a cell stimulation composition, and more particularly a first binding moiety thereof. In one embodiment, the target antigen or epitope thereof is comprised in an entity or in an entity of a complex of entities presented on an external surface of a cell, such as a protein, peptide, or glycoprotein, for example.

In one embodiment, the target cells are mammalian cells. In one embodiment, the target cells are human cells. In one embodiment, the target cells are rodent cells.

In one embodiment, the target cells are lymphocytes, such as T cells, NK cells, or B cells. In one embodiment the target cells are human T cells, NK cells, or B cells. In one embodiment the target cells are mouse T cells, NK cells, or B cells.

In embodiments where the target cells are T cells, the target antigen may be a component of the T cell receptor complex. For example, the component of the T cell receptor complex may be one of CD3 epsilon, CD3 delta, CD3 gamma, CD3 zeta, TCR alpha, TCR beta, TCR gamma, or TCR delta. Thus, binding of the component of the T cell receptor complex by the cell stimulation compositions of this disclosure may elicit a stimulatory response by the T cells or by different T cell subsets, such as an activation of the target T cells which may lead to a proliferation of such T cells or T cell subsets, and/or a secretion of cytokines or chemokines by such T cells or T cell subsets. In addition, the stimulation of one T cell subset, such as CD4′ T cells may in turn result in the stimulation of a different T cell subset, such as CD8′ T cells.

In one embodiment, the target antigen may be a component presented by B cells. For example, the component presented by B cells may include one of CD40, an anti-immunoglobulin, CD79a, or CD79b. Thus, binding of the component of the B cell receptor complex (and subsequent close association of multiple of the components) by the cell stimulation composition(s) of this disclosure may elicit a stimulatory response by the B cells or by different B cell subsets, such as an activation of the target B cells which may lead to a proliferation of such B cells or B cell subsets, and/or a production of antibodies by such B cells or B cell subsets. In addition, the stimulation of one B cell subset may in turn result in the stimulation of a different B cell subset.

In one embodiment, the target antigen may be a component presented by NK cells. For example, the component presented by NK cells may include one of NKp30, NKp44, and NKp46, a C-type lectin-like receptor such as NKG2D, CD335, CD94, CD2, CD16 binding to Fc regions of antibodies, CD122, CD132, IL-15 receptor alpha. Thus, binding of the component presented by the NK cells (and subsequent close association of multiple of the components) by the cell stimulation composition(s) of this disclosure may elicit a stimulatory response by the NK cells or by different NK cell subsets, such as an activation of the target NK cells which may lead to a proliferation of such NK cells or NK cell subsets, and/or a killing of target cells by such NK cells or NK cell subsets. In addition, the stimulation of one NK cell subset may in turn result in the stimulation of a different NK cell subset.

Thus, the methods of stimulating target cells in a sample of cells, comprise contacting the sample of cells with at least one first binding moiety conjugated directly or indirectly to a first nucleic acid polymer (i.e. a cell stimulation composition), the first binding moiety capable of binding a target antigen on the target cells. In one embodiment, the first nucleic acid polymer is engineered with a melting temperature of predicted secondary structures about 60° C. or less (see above for a more detailed description in regard to melting temperatures).

As described above, the first binding moiety may be any type of biological or chemical composition capable of binding a target antigen. Examples of first binding moieties may include an antibody, or a fragment thereof, a peptide, an aptamer, an affimer, or a small molecule.

In one embodiment, the first binding moiety is an antibody, or an antibody fragment. The antibody or fragment thereof may be either monoclonal or polyclonal, but in many cases it is preferred that the antibody or fragment thereof is monoclonal.

A concentration of the first binding moiety conjugated to the first nucleic acid polymer (i.e. cell stimulation composition) used to contact the sample of cells should not be toxic. In one embodiment, the concentration of the cell stimulation composition used to contact the sample of cells is between about 100 μg/mL and 0.001 μg/mL. In one embodiment, the concentration of the cell stimulation composition used to contact the sample of cells is between about 10 μg/mL and 0.01 μg/mL. In one embodiment, the concentration of the cell stimulation composition used to contact the sample of cells is between about 5 μg/mL and 0.05 μg/mL. In one embodiment, the concentration of the cell stimulation composition used to contact the sample of cells is between about 2 μg/mL and 0.02 μg/mL. In one embodiment, the concentration of the cell stimulation composition used to contact the sample of cells is about 0.5 μg/mL or about 1 μg/mL.

The methods also comprise incubating the target cells having been bound by the first binding moiety (e.g. the soluble cell stimulation composition).

A duration of the incubation of target cells having been bound by the first binding moiety will usually depend on the nature of the target cells. For example, if the target cells are T cells, the T cells having been bound by the cell stimulation composition (more specifically, by the first binding moiety thereof), and any necessary or desired second binding moiety or co-stimulatory binding moiety should be incubated for any amount of time that does not negatively impact the cells. In some embodiments, it may be appropriate to incubate the T cells having been bound by the first binding moiety for up to 14 days. In one embodiment, the bound T cells are incubated for less than 14 days, such as 13 days, 12 days, 11 days, 10 days, 9 days, 8 days, 7 days, 6 days, 5 days, 4 days, 3 days, 2 days or 1 day. In a preferred embodiment, the T cells having been bound by the first binding moiety are incubated for about 2 days or about 3 days.

If the target cells are B cells, the B cells having been bound by the cell stimulation composition (more specifically, by the first binding moiety thereof), and any necessary or desired co-stimulatory binding moiety or accessory signal should be incubated for any amount of time that does not negatively impact the cells. In some embodiments, it may be appropriate to incubate the B cells having been bound by the at least one first binding moiety for 2 weeks, for 1 week, for 6 days, for 5 days, for 4 days, for 3 days, for 2 days, or for 1 day, or less.

If the target cells are NK cells, the NK cells having been bound by the cell binding composition (more specifically, by the first binding moiety thereof), and any necessary or desired co-stimulatory binding moiety or accessory signal should be incubated for any amount of time that does not negatively impact the cells. In some embodiments, it may be appropriate to incubate the NK cells having been bound by the at least one first binding moiety for 2 weeks, for 1 week, for 6 days, for 5 days, for 4 days, for 3 days, for 2 days, or for 1 day, or less.

Since stimulating target cells in a sample of cells was surprisingly shown using first binding moieties conjugated to first nucleic acid polymers (i.e. cell stimulation compositions) without the need to pre-complex two of such conjugates to one another, it may be important to modify or engineer a sequence of the first nucleic acid polymer such that a melting temperature of predicted secondary structures thereof is about 60° C. or less. In one embodiment, the first nucleic acid polymer comprises a sequence wherein melting temperature of predicted secondary structures is between about 50° C. and 10° C., or less. In one embodiment, the first nucleic acid polymer comprises a sequence wherein melting temperature of predicted secondary structures is between about 40° C. and 20° C.

A not necessarily mutually exclusive feature of the first nucleic acid polymer may relate to its free energy. In one embodiment, a predicted free energy of the first nucleic acid polymer at 150 mM monovalent ion concentration is between about +1 kcal/mol and −10 kcal/mol. In one embodiment, a predicted free energy of the first nucleic acid polymer at 150 mM monovalent ion concentration is negative.

Depending on the application of the cell stimulation compositions, it may be desirable that a sequence of the first nucleic acid polymer is devoid of motifs known to trigger cellular responses, such as stimulatory responses. For example, a first nucleic acid polymer rich in CpG motifs may elicit a pro-inflammatory response via toll-like receptor (TLR) 9 recognition. Alternatively, double-stranded DNA polymers with unpaired ends (Y-form) containing guanosines may also be recognized via the cytosolic immune-sensing receptor cGAS or via AIM-1 in a sequence-independent fashion for fragments ranging between 50 to 80 bp. Furthermore, tri-phopshorylated (5′ppp) double-stranded ribonucleic acid (RNA) polymers can also elicit immune responses via RIG-1 or via TLR3 when longer than 40 bp. Those containing polyU or G/U-rich sequences may also be recognized by TLR7, for example. Single-stranded RNA polymers containing polyU/UC motifs may also elicit immune response via RIG-1 signaling.

Another important feature of the first nucleic acid polymer may be a length thereof. Since the possibility of secondary structures usually increases with the length of a single stranded first nucleic acid polymer, in most embodiments the first nucleic acid polymer is about 100 monomers long, or less. In one embodiment, the first nucleic acid polymer is about 90 monomers long, or less. In one embodiment, the first nucleic acid polymer is about 80 monomers long, or less. In one embodiment, the first nucleic acid polymer is about 70 monomers long, or less. In one embodiment, the first nucleic acid polymer is about 60 monomers long, or less. In one embodiment, the first nucleic acid polymer is about 50 monomers long, or less. In one embodiment, the first nucleic acid polymer is about 40 monomers long, or less. In one embodiment, the first nucleic acid polymer is about 30 monomers long, or less. In one embodiment, the first nucleic acid polymer is about 20 monomers long, or less. In one embodiment, the first nucleic acid polymer is at least about 10 monomers long. In one embodiment, the first nucleic acid polymer is at least about 5 monomers long.

In one embodiment, the methods may optionally comprise contacting the sample of cells with at least one second binding moiety and/or a co-stimulatory binding moiety capable of binding a second target antigen and/or a co-stimulatory antigen on the target cells, either before, contemporaneous with the at least one first binding moiety (conjugated to the first nucleic acid polymer), or after contacting the sample of cells with the at least one first binding moiety (conjugated to the first nucleic acid polymer).

Similar to the first binding moiety, the second binding moiety and/or a co-stimulatory binding moiety may be an antibody, or a fragment thereof, a peptide, an aptamer, an affimer, or a small molecule. Likewise to the first binding moiety, the second binding moiety and/or the co-stimulatory binding moiety may either be soluble or immobilized on a surface. In one embodiment, the second binding moiety and/or co-stimulatory is soluble. In one embodiment, both the cell stimulation composition and the second binding moiety and/or the co-stimulatory binding moiety are soluble.

A concentration of the second binding moiety (and/or the co-stimulatory binding moiety) used to contact the sample of cells should not be toxic. In one embodiment, the concentration of the second binding moiety (and/or the co-stimulatory binding moiety) used to contact the sample of cells is between about 100 μg/mL and 0.001 μg/mL. In one embodiment, the concentration of the second binding moiety (and/or the co-stimulatory binding moiety) used to contact the sample of cells is between about 10 μg/mL and 0.01 μg/mL. In one embodiment, the concentration of the second binding moiety (and/or the co-stimulatory binding moiety) used to contact the sample of cells is between about 5 μg/mL and 0.05 μg/mL. In one embodiment, the concentration of the second binding moiety (and/or the co-stimulatory binding moiety) used to contact the sample of cells is between about 2 μg/mL and 0.02 μg/mL. In one embodiment, the concentration of the second binding moiety (and/or the co-stimulatory binding moiety) used to contact the sample of cells is about 0.5 μg/mL or about 1 μg/mL.

A duration of the incubation of target cells having been bound by the first binding moiety will usually depend on the nature of the target cells. For example, if the target cells are T cells, the T cells having been bound by the cell stimulation composition (more specifically, by the first binding moiety thereof), and any necessary or desired second binding moiety and/or costimulatory binding moiety should be incubated for any amount of time that does not negatively impact the cells. In some embodiments, it may be appropriate to incubate the T cells having been bound by the first binding moiety for up to 14 days. In one embodiment, the bound T cells are incubated for less than 14 days, such as 13 days, 12 days, 11 days, 10 days, 9 days, 8 days, 7 days, 6 days, 5 days, 4 days, 3 days, 2 days or 1 day. In a preferred embodiment, the T cells having been bound by the first binding moiety are incubated for about 2 days or about 3 days.

If the target cells are B cells, the B cells having been bound by the cell stimulation composition (more specifically, by the first binding moiety thereof), and any necessary or desired second binding moiety and/or costimulatory binding moiety should be incubated for any amount of time that does not negatively impact the cells. In some embodiments, it may be appropriate to incubate the B cells having been bound by the at least one first binding moiety for 2 weeks, for 1 week, for 6 days, for 5 days, for 4 days, for 3 days, for 2 days, or for 1 day, or less.

If the target cells are NK cells, the NK cells having been bound by the cell binding composition (more specifically, by the first binding moiety thereof), and any necessary or desired second binding moiety and/or costimulatory binding moiety should be incubated for any amount of time that does not negatively impact the cells. In some embodiments, it may be appropriate to incubate the NK cells having been bound by the at least one first binding moiety for 2 weeks, for 1 week, for 6 days, for 5 days, for 4 days, for 3 days, for 2 days, or for 1 day, or less.

In one embodiment, the methods may further comprise increasing (or increased) proliferation of the target cells during the incubation step, wherein proliferation of the target cells is greater in the presence of the cell stimulation composition (and any necessary co-stimulatory molecule(s)) than in the absence of the cell stimulation composition (and any necessary co-stimulatory molecule(s)).

In one embodiment, increased proliferation is dependent on contacting the sample of cells with both at least one first binding moiety conjugated to a first nucleic acid polymer and at least one second binding moiety capable of binding a second target antigen on the target cells (and/or and at least one co-stimulatory binding moiety capable of binding a co-stimulatory antigen on the target cells). For example, if the target cells are T cells then the first target antigen may be a component of the T cell receptor complex, such as CD3epsiolon, TCRα, or TCRβ, and the second target antigen (and/or co-stimulatory antigen) may be one or more of: costimulatory receptors of the immunoglobulin superfamily including CD28, CD137, CD2, CD134, CD152, CD278, CD27, or CD279, or a different tumour necrosis receptor family member. In one embodiment, an additional signal, such as a cytokine, may help boost proliferation, activation, or modulation of activity.

In embodiments where the target cells are B cells, then the first antigen may be one or more of CD40, an anti-immunoglobulin, CD79a, or CD79b, and the second target antigen (and/or co-stimulatory antigen) may be a different one or more of CD40, an anti-immunoglobulin, CD79a, or CD79b. In one embodiment, a signal such as one or more of IL-4, IL-6, IL-10 and IL-13 (for human B cells), may be used together with or in place of binding the second target antigen (and/or the co-stimulatory antigen).

In embodiments where the target cells are NK cells, then the first antigen may be one of NKp30, NKp44, and NKp46, and C-type lectin-like receptors, such as NKG2D, CD335, CD94, CD2, CD16 binding to Fc regions of antibodies, CD122, CD132, IL-15 receptor alpha, and the second antigen (and/or the co-stimulatory antigen) may be a different one or more of NKp30, NKp44, and NKp46, and C-type lectin-like receptors, such as NKG2D, CD335, CD94, CD2, CD16 binding to Fc regions of antibodies, CD122, CD132, IL-15 receptor alpha. In one embodiment, a signal such as one or more of IL-2, IL-12, IL-15, IL-18, and IL-21, may be used together with or in place of binding the second target antigen (and/or the co-stimulatory antigen).

In one embodiment, the first nucleic acid polymer or a complementarity region thereof is complementary to a second nucleic acid polymer. The second nucleic acid polymer may either be directly or indirectly conjugated to at least one second binding moiety, or not conjugated to at least one second binding moiety.

In embodiments where the second nucleic acid polymer is not conjugated to at least one second binding moiety, the methods disclosed herein may further comprise hybridizing a second nucleic acid polymer to the first nucleic acid polymer or a complementarity region thereof. In hybridizing the second nucleic acid polymer to the first nucleic acid polymer, or the complementarity region thereof, either before or after contacting the sample of cells with the cell stimulation composition, it may be possible to modulate a level of target cell stimulation. Put another way, activity of the first binding moiety conjugated to the first nucleic acid polymer may be quenched when the first nucleic acid polymer, or complementarity region thereof, is hybridized with the second nucleic acid polymer.

In one embodiment, the second nucleic acid polymer may be pre-hybridized to the first nucleic acid polymer (or the complementarity region thereof) before contacting the sample of cells with the first binding moiety conjugated directly or indirectly to a first nucleic acid polymer. In such an embodiment, stimulating the target cells in the sample of cells may be initially quenched, and therefore stimulating the target cells may be inducible. For example, stimulating the target cells may be induced at an appropriate time by adding a competitor nucleic acid polymer, wherein a hybridization preference of the second nucleic acid polymer for the competitor nucleic acid polymer is higher than for the first nucleic acid polymer (or the complementarity region thereof).

In one embodiment, the second nucleic acid polymer may be hybridized to the first nucleic acid polymer (or the complementarity region thereof) contemporaneous with or after contacting the sample of cells with the soluble first binding moiety conjugated directly or indirectly to a first nucleic acid polymer. In such an embodiment, stimulating the target cells in the sample of cells may be initially induced, and stimulating the target cells may subsequently be uninduced. For example, stimulating the target cells may be uninduced at an appropriate time by hybridizing a second nucleic acid polymer to the first nucleic acid polymer (or the complementarity region thereof). And, at a later appropriate time reactivating target cell stimulation may be achieved by adding a competitor nucleic acid polymer, wherein a hybridization preference of the second nucleic acid polymer for the competitor nucleic acid polymer is higher than for the first nucleic acid polymer (or the complementarity region thereof).

By way of example, to illustrate the concept behind inducing and uninducing the stimulation of target cells in a sample of cells using a first binding moiety conjugated to a first nucleic acid polymer (i.e. cell stimulation composition), a second nucleic acid polymer, and in applicable embodiments, a competitor nucleic acid polymer. The second nucleic acid polymer may be 20 monomers long, and such second nucleic acid polymer may be about 40%, 50%, 60%, 70%, 80% or 90% complementary to the first nucleic acid polymer, or a complementarity region thereof. Thus, the second nucleic polymer has sufficient complementarity to hybridize to the first nucleic acid polymer, or the complementarity region thereof. However, in embodiments including a competitor nucleic acid polymer, the complementarity between the second nucleic acid polymer and the competitor nucleic acid polymer is higher than is the complementarity between the second nucleic acid polymer and the first nucleic acid polymer, or the complementarity region thereof. For example, if the second nucleic acid polymer is 50% complementary to the first nucleic acid polymer, or complementarity region thereof, then a competitor nucleic acid polymer that is about 60%, 70%, 80%, 90%, or 100% complementary to the second nucleic acid polymer will have greater hybridization preference for the second nucleic acid polymer than the hybridization preference of the second nucleic acid polymer for the first nucleic acid polymer, or the complementarity region thereof.

In embodiments where the second nucleic acid polymer is directly or indirectly conjugated to either a first binding moiety and/or a second binding moiety, the conjugation may be performed in any way known to or accessible by the person skilled in the art. For example, a first or second binding moiety may be conjugated to the second nucleic acid polymer using any known reaction, provided that the constitution of these components is not negatively impacted during the conjugation reaction(s). In one embodiment, the second nucleic acid polymer may be conjugated to the first or second binding moiety by one of nucleophilic substitution, electrophilic substitution, alkylation, oxidation, condensation, cycloaddition or Staudinger ligation reaction.

In one embodiment, the second nucleic acid polymer may be conjugated to the first and/or second binding moiety by one or more covalent bond linkages involving hydrazone, oxime, imides, sulfones, triazine, or other covalent attachment via functional groups selected from the group comprising sulfhydryl, amine, carboxyl, hydroxyl, hydrazide, aldehyde or glycans.

In such embodiments, where the second nucleic acid polymer is conjugated to a first and/or second binding moiety, the methods disclosed herein may further comprise hybridizing such second nucleic acid polymer to the first nucleic acid polymer or a complementarity region thereof. In hybridizing the second nucleic acid polymer to the first nucleic acid polymer or the complementarity region thereof, either before, contemporaneous with, or after contacting the sample of cells with the soluble second binding moiety, it may be possible to form a bispecific complex of binding moieties. In such embodiments, the bispecific complex of binding moieties is a cell stimulation composition.

In one embodiment, the first binding moiety and the second binding moiety bind the same target antigen. In one embodiment, the first binding moiety and the second binding moiety bind different target antigens.

In one embodiment, the second nucleic acid polymer is pre-hybridized to the first nucleic acid polymer (or the complementarity region thereof) prior to the bispecific complex of binding moieties coming into contact with the sample of cells (and the target cells therein). In such an embodiment, the bispecific complex of binding moieties may initially elicit the stimulatory response of the target cells. At an appropriate time the stimulatory response of the target cells to the bispecific complex of binding moieties may be quenched with a competitor nucleic acid polymer, wherein a hybridization preference of the competitor nucleic acid polymer for the first nucleic acid polymer (or the complementarity region thereof) or the second nucleic acid polymer (or the complementarity region thereof) is higher than a hybridization preference of the first nucleic acid polymer for the second nucleic acid polymer. At a later appropriate time the stimulatory response may be reactivated with an anti-competitor nucleic acid polymer, wherein a hybridization preference of the anti-competitor nucleic acid polymer for the competitor nucleic acid polymer is higher than a hybridization preference of the competitor nucleic acid polymer for the first nucleic acid polymer (or the complementarity region thereof) or the second nucleic acid polymer (or the complementarity region thereof).

In one embodiment, formation of the bispecific complex of binding moieties may initially be perturbed by hybridization of a competitor nucleic acid polymer to either the first nucleic acid polymer or second nucleic acid polymer, or both, wherein a hybridization preference of the competitor nucleic acid polymer for the first nucleic acid polymer and/or the second nucleic acid polymer is higher than a hybridization preference of the first nucleic acid polymer for the second nucleic acid polymer. Thus, the stimulatory response of the target cells may initially be quenched. Accordingly, the individual components are not able to form a bispecific complex of binding moieties, but a bispecific complex of binding moieties may form by adding, at an appropriate time, an anti-competitor nucleic acid polymer, wherein a hybridization preference of the anti-competitor nucleic acid polymer for the competitor nucleic acid polymer is higher than a hybridization preference of the competitor nucleic acid polymer for the first nucleic acid polymer and/or the second nucleic acid polymer.

By way of example, to illustrate the concept behind inducing and uninducing the stimulation of target cells in a sample of cells using: a first binding moiety conjugated to a first nucleic acid polymer; a (a different first or) second binding moiety conjugated to a second nucleic acid polymer; and in applicable embodiments, a competitor nucleic acid polymer and an anti-competitor nucleic acid polymer. The second nucleic acid polymer may be about 40%, 50%, 60%, 70%, or 80% complementary to the first nucleic acid polymer, or a complementarity region thereof. Thus, the second nucleic polymer has sufficient complementarity to hybridize to the first nucleic acid polymer, or the complementarity region thereof. However, in embodiments including a competitor nucleic acid polymer, the complementarity between the first nucleic acid polymer or the second nucleic acid polymer and the competitor nucleic acid polymer is higher than is the complementarity between the first nucleic acid polymer, or the complementarity region thereof, and the second nucleic acid polymer. For example, if the second nucleic acid polymer is 50% complementary to the first nucleic acid polymer, or complementarity region thereof, then a competitor nucleic acid polymer that is about, for example, 60% or 70% complementary to the first nucleic acid polymer, or complementarity region thereof, or second nucleic acid polymer will have greater hybridization preference for the first nucleic acid polymer, or complementarity region thereof, or second nucleic acid polymer than the hybridization preference of the second nucleic acid polymer for the first nucleic acid polymer, or the complementarity region thereof. Further, if an anti-competitor nucleic acid polymer is 80% or 90% or 100% complementary to the competitor nucleic acid polymer, then the anti-competitor nucleic acid polymer will have greater hybridization preference for the competitor nucleic acid polymer than the hybridization preference of the competitor nucleic acid polymer for the first nucleic acid polymer, or complementarity region thereof, or the second nucleic acid polymer. Therefore, with the competitor nucleic acid polymer hybridized to the anti-competitor nucleic acid polymer, the second nucleic acid polymer may hybridize to the first nucleic acid polymer, or the complementarity region thereof.

If there is a concern in applications of use that the uncomplexed components of the bispecific complex of binding moieties may individually trigger the stimulatory response in the target cells, this may be controlled in accordance with the teachings herein. For example, a sequence of the first and/or second nucleic acid polymers may be engineered to have disabling secondary structures (i.e. having melting temperatures above 50° C. or 60° C., or higher). By way of additional example, the uncomplexed components of the bispecific complex of binding moieties may be “neutralized” or “disabled” with appropriate neutralizing or disabling nucleic acid polymers that hybridize to the first and second nucleic acid polymers.

Accordingly, although the cell stimulation compositions of this disclosure were surprisingly shown to stimulate target cells without the need to pre-complex two of such cell stimulation compositions to one another, it may nevertheless be desirable in some cases.

In one embodiment, the target antigen bound by the first binding moiety and the target antigen bound by the second binding moiety are on the same cell. In one embodiment, the target antigen bound by the first binding moiety and the target antigen bound by the second binding moiety are on different cells. In such embodiments, the systems and methods described above may be adapted to bring two target cells types into proximity of one another on demand. For example, a first binding moiety may be bound to a first target cell type and a second binding moiety may be bound to a second target cell type, and the otherwise complementary respective nucleic acid polymers of such first binding moiety and such second binding moiety may each be hybridized to competitor nucleic acid polymers. Upon addition of appropriate anti-competitor nucleic acid polymers, competitor:anti-competitor duplexes may form allowing the respective (first and second) nucleic acid polymers to hybridize, thereby bringing the first target cell type and the second target cell type into proximity.

The following non-limiting examples are illustrative of the present disclosure.

TABLE 1
Listing of nucleic acid polymers and the corresponding sequences used in the
disclosure.

| Name | Length | Sequence (5′→3′)ª |
|---|---|---|
| A | 69 | CCTTGGCACCCGAGAATTCCACTCATTGTAACTCCTNAAAAAAAAAAAAAAAAAAAAAAA |
| | | AAAAAAA*A*A (SEQ ID NO: 1) |
| AC1 | 36 | AGGAGTTACAATGAGTGGAATTCTCGGGTGCCAAGG (SEQ ID NO: 2) |
| A_var1 | 40 | CCTTGGCACCCGAGAATTCCATATCCCTTGGGATGGAAAA (SEQ ID NO: 3) |
| A_var2 | 40 | CCTTTGCACCCGAGAATTCCATATCCCTTGTGATAGAAAA (SEQ ID NO: 4) |
| B | 90 | GTGACTGGAGTTCAGACGTGTGCTCTTCCGATCTNNNNNNNNNNCTCATTGTAACTCCT |
| | | NNNNNNNNNGCTTTAAGGCCGGTCCTAGC*A*A (SEQ ID NO: 5) |
| B var1 | 70 | GTGACTGGAGTTCAGACGTGTGCTCTTCCGATCCTCATTGTAACTCCTGCTTTAAGGCCG |
| | | GTCCTAGCAA (SEQ ID NO: 6) |
| B_var2 | 71 | GTGATTGTAGTTCAGATGTGTGCTCTTCCGATCTCTCATTGTAACTCCTTCTTTAAGTCCG |
| | | GTCCTAGCAA (SEQ ID NO: 7) |
| C | 71 | CGGAGATGTGTATAAGAGACAGNNNNNNNNNNCTCATTGTAACTCCTNNNNNNNNNC |
| | | CCATATAAGA*A*A (SEQ ID NO: 8) |
| C_var1 | 50 | CGGAGATGTGTATAAGAGACAGCTCATTGTAACTCCTCCCATATAAGAAA (SEQ ID |
| | | NO: 9) |
| C_var2 | 69 | CGGAGATGTGTATAAGAGACAGTTTTTTTTTTCTCATTGTAACTCCTTTTTTTTTTCCCATA |
| | | TAAGAAA (SEQ ID NO: 10) |
| D | 35 | GGCTTTTACTTCTATCTCTTTCTTTAACACTTTCT (SEQ ID NO: 11) |
| DC | 44 | GAAAGTGTTAAAGAAAGAGATAGAAGTAAAAGCCTCGATACGTA (SEQ ID NO: 12) |
| E | 69 | CCTTGGCACCCGAGAATTCCATATCCCTTGGGATGGNAAAAAAAAAAAAAAAAAAAAAA |
| | | AAAAAAAAAA (SEQ ID NO: 13) |
| F | 69 | CCTTGGCACCCGAGAATTCCACGTAACGTAGAGCGANAAAAAAAAAAAAAAAAAAAAA |
| | | AAAAAAAAAAA (SEQ ID NO: 14) |
| CC1 | 22 | CTGTCTCTTATACACATCTCCG (SEQ ID NO: 15) |
| CC12 | 40 | AGGAGTTACAATGAGTTTCTGTCTCTTATACACATCTCCG (SEQ ID NO: 16) |
| CC3 | 13 | TTTCTTATATGGG (SEQ ID NO: 17) |
| CC23 | 31 | TTTCTTATATGGGTTTAGGAGTTACAATGAG (SEQ ID NO: 18) |
| C2x | 29 | CCCATATAAGAAATTTAAAGAATATACCC (SEQ ID NO: 19) |
| C3x | 45 | CCCATATAAGAAATTTAAAGAATATACCCTTTCCCATATAAGAAA (SEQ ID NO: 20) |
| C4x | 61 | CCCATATAAGAAATTTAAAGAATATACCCTTTCCCATATAAGAAATTTAAAGAATATACCC |
| | | (SEQ ID NO: 21) |

^{a}N designates a random nucleotide (A, C, G, or T); * designates a phosphorothioate bond

TABLE 2
Properties of the compositions and nucleic acid polymers thereof used in this disclosure.

| Composition | Conjugate/ | Secondary structure^{b} | Secondary structure | T cell Activation |
|---|---|---|---|---|
| Name | Antibody | deltaG (Kcal mol⁻¹) | Tm (° C.) | outcome |
| A | N/A/CD3 clone 1 | −1.18 to −1.03 | 33.6 to 38.2 | +++ |
| | (BioLegend) | | | |
| A_var1 | Thiol/CD3 clone 1 | −5.87 to −3.01 | 42.3 to 51.6 | + |
| A_var2 | Thiol/CD3 clone 1 | −0.53 to −0.45 | 21.8 to 31.0 | +++ |
| B | N/A/CD3 clone 1 | −4.23 to −2.27 | 31.7 to 45.2 | + |
| | (BioLegend) | | | |
| B_var1 | Thiol/CD3 clone 1 | −5.36 to −2.75 | 31.9 to 44.5 | + |
| B_var2 | Thiol/CD3 clone 1 | −0.85 to 0.14 | 24.1 to 28.1 | +++ |
| C | N/A/CD3 clone 1 | −0.54 to 0.44 | 21.7 to 29.2 | +++ |
| | (BioLegend) | | | |
| C_var1 | Thiol/CD3 clone 1 | −3.37 to −1.73 | 39.5 to 48.9 | + |
| C_var2 | Thiol/CD3 clone 1 | −1.63 to −0.65 | 27.9 to 33.5 | +++ |
| D | Amine/CD3 clone 1 | −4.47 to −2.32 | 38.4 to 60.8 | + |
| DC | Amine/clone 1 | 0.07 to 0.95 | −1.3 to 24 | + |
| E | N/A/CD3 clone 2 | −5.11 to −3.01 | 42.3 to 51.6 | ++ |
| | (BioLegend) | | | |
| F | N/A/TCRαβ clone | −3.26 to −2.01 | 38.7 to 45.2 | + |
| | 1 (BioLegend) | | | |
| C2x | Thiol/CD3 clone 1 | −0.25 to 0.51 | 17.3 to 30.6 | + |
| C3x | Thiol/CD3 clone 1 | −1.04 to −0.05 | 25.3 to 31.5 | ++ |
| C4x | Thiol/CD3 clone 1 | −1.29 to −0.39 | 27.5 to 32.2 | +++ |

^{b}secondary structure predictions reported for 150 mM monovalent cation concentration at 25° C.

### EXAMPLES

### Example 1: Conjugating Nucleic Acid Polymers to Binding Moieties

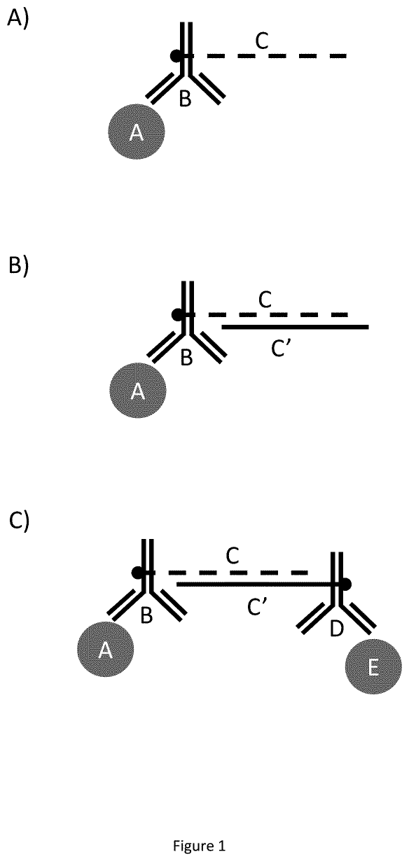

Nucleic acid polymer-conjugated (monoclonal) antibodies are generally depicted as in FIG. 1. Such conjugates may be produced via chemical reactions known in the art, including an amine-based and a thiol-based conjugation approach.

The amine-based covalent attachment of DNA oligo sequences to a monoclonal anti-human antibody (mAb) of this disclosure proceeds via an intermediate dibenzocyclooctyne-PEG4-N-Hydroxysuccinimide (DBCO-PEG4-NHS Ester; Click Chemistry Tools cat no: A134) linker molecule. The following general description is also applicable to other antibodies and alternative nucleic acid polymer sequences, such as those presented in Table 1. Compositions used in the disclosure are listed in Table 2.

A 5 mg/mL stock solution of DBCO-PEG4-NHS linker was prepared in DMSO. A solution of anti-CD3 mAb (clone 1; STEMCELL Technologies) in 1×PBS was then mixed with 180 mol excess of linker and 1×PBS to achieve a final mAb concentration of 1 mg/mL and an effective DMSO concentration that is below 10%. The preparation was thoroughly mixed and incubated at room temperature for 90 minutes.

Following mAb modification, the reaction was purified by ultrafiltration (e.g. Amicon 30K MWCO, EMD Millipore cat no: UFC503024) by exchanging 3× in 1×PBS to remove unreacted linker. The purified product was immediately analyzed by UV-Vis spectroscopy, wherein antibody concentration and degree of modification was determined by measuring absorbance at 280 nm and 310 nm and calculating using a known molar extinction coefficient for both IgG and DBCO. A degree of substitution of <4 DBCO per IgG is preferred. Two to four mol equivalents of azide-modified DNA nucleic acid polymers (Integrated DNA technologies Inc.) (resuspended in 1×PBS) were added to the modified antibody preparation and incubated overnight (16-24 hrs) at 4° C.

Following the amine-based conjugation reaction, the conjugates are purified from unreacted free oligos via FPLC using a Ni²⁺ loaded immobilized metal affinity chromatography column (HiTrap Chelating HP, GE healthcare cat no: 17040801). First, 0.5 column volume (CV) of 0.1M NiCl₂ in water was loaded onto the column according to the manufacturer's specifications. After thorough washing with 5 CV of running buffer (0.1M sodium phosphate pH 8.0, 300 mM NaCl), the conjugates were injected and allowed to bind to the column at a flow rate of 1 ml/min. After washing the column with 10 CV of running buffer, retained conjugates were eluted with running buffer containing 50 mM EDTA. Collected fractions containing the eluted conjugates were immediately processed by ultrafiltration (e.g. Amicon 30K MWCO, EMD Millipore cat no UFC503024) and exchanged in 1×PBS, 2 mM EDTA for storage at 4° C.

The thiol-based covalent attachment of DNA oligo sequences to a monoclonal anti-human antibody (mAb) of this disclosure proceeds via an intermediate dibenzocyclooctyne-PEG4-bis-sulfone (DBCO-PEG4-bis-sulfone; Click Chemistry Tools cat no: 1144) linker. The following general description provided is also applicable to other antibodies and alternative nucleic acid polymer sequences, such as those presented in Table 1. Compositions used in the disclosure are listed in Table 2.

A 2 mg/mL stock solution of DBCO-PEG4-bis-sulfone linker was prepared in DMSO. One hundred nmol of linker is combined with 5 nmol azide-modified oligo (Integrated DNA technologies Inc.) and DMSO is added to adjust to a final solvent concentration >60%. The preparation was thoroughly mixed and incubated at room temperature for 3 hrs. Following oligo modification, an appropriate volume of 1×PBS was added to reduce the DMSO concentration below 30%. Upon mixing, a portion of unreacted linker will precipitate and the reaction will appear cloudy. After spinning the preparation at 10,000 g for 2 mins, the supernatant is collected and purified by ultrafiltration (Amicon 10K MWCO, EMD Millipore cat no: UFC501024) by exchanging 4× in 1×PBS. The purified oligos were immediately analyzed by UV-Vis spectroscopy (Nanodrop) by measuring absorbance at 260 nm.

A solution of anti-CD3 mAb (clone 1; STEMCELL Technologies Inc.) in 1×PBS was mixed with 5 mM TCEP at a final concentration of 1 mg/mL, and incubated at room temperature for 30 mins to reduce interchain disulfides.

Following thiol-based mAb reduction, the reaction was purified by ultrafiltration (Amicon 10K MWCO, EMD Millipore, cat no. UFC501024) by exchanging 3× in 1×PBS, 2 mM EDTA to remove TCEP. The purified product was immediately analyzed by UV-Vis spectroscopy, to determine antibody concentration by measuring absorbance at 280 nm. 2 to 4 mol equivalents of previously modified DNA nucleic acid polymers were added to the reduced antibody preparation and incubated overnight (16-24 hrs) at 4° C. Following the conjugation reaction, the conjugates are purified as per the previous description.

### Example 2: Preparation of Cells

Target cells were enriched from a sample by negative selection. For stimulation experiments, T cells were isolated from fresh PBMCs using an immunomagnetic negative T cell isolation approach (EasySep™ Human T Cell Isolation Kit, STEMCELL Technologies, cat no. 17951) according to the manufacturer's instructions.

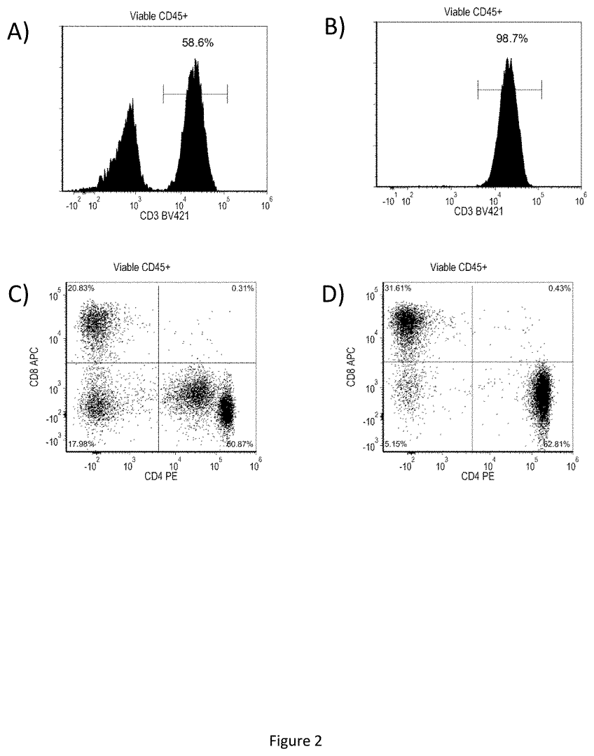

Following the isolation protocol, the concentration of enriched cells was determined using standard approaches. Optionally, the purity of the negatively selected cells may be determined by flow cytometry (FIG. 2). Depending on the donor, it is typical that among viable CD45+ cells in a PBMC sample, that approximately 60% of such cells are CD3+(FIG. 2A). After enriching for T cells in accordance with this Example, it is typical to obtain a population of viable CD45+ cells comprising 95% purity of CD3+ T cells (FIG. 2B). FIGS. 2C (pre-enrichment) and 2D (post-enrichment) show flow cytometry plots of various T cells subsets expressing either CD4 or CD8, or both, and confirm the retention of the subset populations following isolation.

### Example 3: Binding Target Cells Using Cell Stimulation Compositions

The ability of the compositions made in accordance with Example 1 (unless commercially purchased pre-conjugated) to bind the enriched cells of Example 2 was tested.

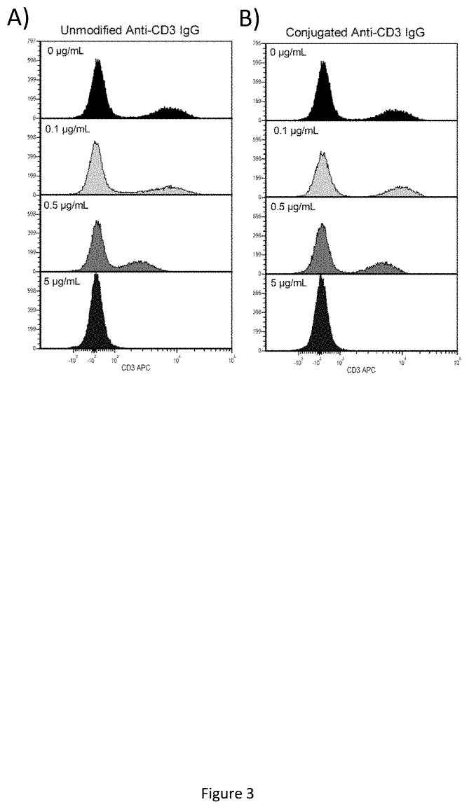

For T cells, it is shown in FIG. 3 that compositions of this disclosure are capable of binding enriched T cells. Briefly, 1.6×10⁵ cells were seeded per well of a 96-well plate and incubated with 0 μg/mL, 0.1 μg/mL, 0.5 μg/mL, or 5 μg/mL of either unmodified anti-human CD3 IgG (clone 1) (FIG. 3A) or anti-human CD3 IgG (Clone 1) conjugated with oligonucleotide A (FIG. 3B). After incubating the cells and antibody compositions at room temperature for approximately 20 minutes, a fixed concentration (0.5 μg/mL) of APC-labeled anti-human CD3 IgG (Biolegend) was added to compete for available antigen binding sites on the T cells. Following incubation with the APC-labeled anti-human CD3 IgG antibodies, the samples were washed by successive rounds of centrifugation and resuspension in staining buffer. The labelled cells were subsequently analyzed by flow cytometry (CytoFLEX, Beckman Coulter). A dose-dependent antigen blocking with unlabeled anti-human CD3 IgG (clone 1) or anti-human CD3 IgG (Clone 1) oligo conjugates is observed, indicating that the compositions of this disclosure bind target cells.

### Example 4: Stimulating Isolated Target Cells with Cell Stimulation Compositions and Costimulatory Molecules

The ability of the compositions made in accordance with Example 1 (unless commercially purchased pre-conjugated) to stimulate a response in the enriched cells of Example 2 was tested.

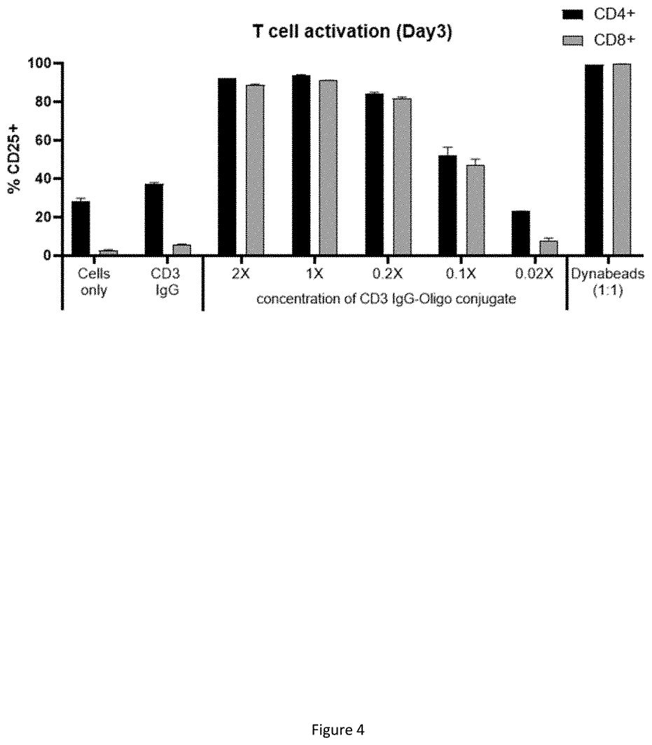

For human T cells, it is shown in FIG. 4 that compositions of this disclosure are capable of stimulating T cells. Briefly, 1.6×10⁵ cells were seeded per well of a 96-well plate and contacted with various compositions to test for % CD25⁺ expression following a 72-hour incubation period at 37° C. in humidified 5% CO₂ atmosphere. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2) (CD3 IgG only condition). A third control condition also included Dynabeads™ (Dynabeads™ Human T activator CD3/CD28; ThermoFisher cat no. 1131D) incubated at a 1:1 bead per cell ratio. Treatment conditions were investigated with 2×, 1×, 0.2×, 0.1×, or 0.02× concentrations of anti-human CD3 IgG (Clone 1) conjugated with oligonucleotide A in the presence of 1 μg/mL anti-human CD28 IgG for the duration of the 72-hour incubation period at 37° C. Higher concentrations of the soluble cell stimulation composition (i.e. anti-human CD3 IgG (Clone 1) conjugated to oligonucleotide A) stimulated both CD4⁺ T cells (black bars) and CD8⁺ T cells (grey bars) to comparable levels obtained with Dynabeads™. Furthermore, the conjugation of the nucleic acid polymer was important for stimulation, since unconjugated anti-human CD3 IgG (Clone 1) failed to elicit a stimulatory response.

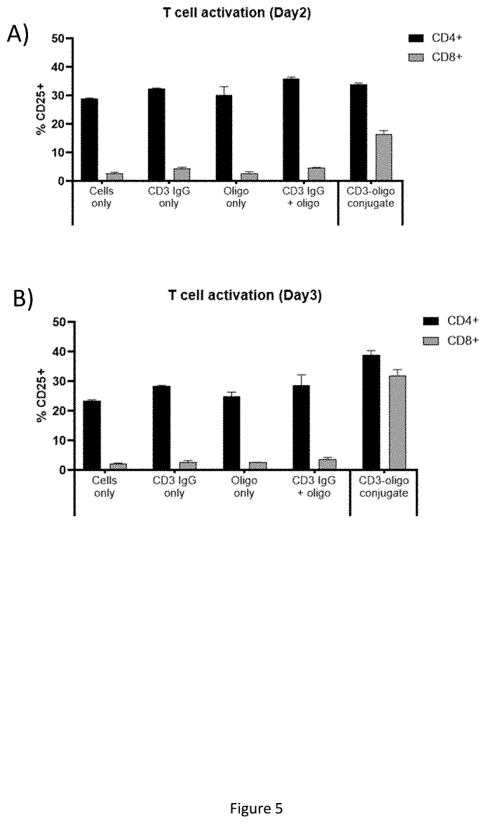

For human T cells, it is shown in FIG. 5 that compositions of this disclosure capably stimulate T cells when binding moieties and nucleic acid polymers are conjugated to one another. The unconjugated form elicits low or no stimulatory response. Briefly, 1.6×10⁵ cells were seeded per well of a 96-well plate and contacted with various compositions to test for % CD25+ expression following either a 48-hour (FIG. 5A) or a 72-hour (FIG. 5B) incubation period at 37° C. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2) (CD3 IgG only condition). A third control condition included the nucleic acid polymer alone in the presence of CD28 IgG (oligo only condition). Seeded cells were treated with either co-incubation of 0.5 μg/mL unmodified CD3 IgG with 1 μM soluble nucleic acid polymer D in the presence of 1 μg/mL CD28 IgG, or with 0.5 μg/mL CD3 IgG:oligonucleotide D conjugate in the presence of 1.0 μg/mL CD28 IgG. Following the 48 or 72-hour incubation period, cells were analyzed for CD25 activation marker expression. Only the condition where anti-human CD3 IgG (clone 1) was conjugated with oligonucleotide D resulted in a stimulatory response.

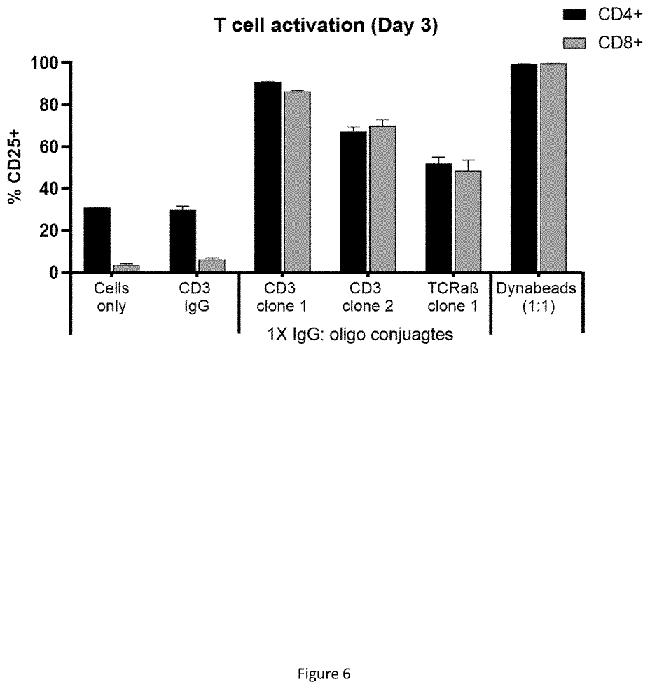

For human T cells, it is shown in FIG. 6 that compositions of this disclosure are capable of stimulating T cells. Briefly, 1.6×10⁵ cells were seeded per well of a 96-well plate and contacted with various compositions to test for % CD25+ expression following a 72-hour incubation period at 37° C. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2) (CD3 IgG condition). A third control condition also included Dynabeads™ incubated at a 1:1 bead per cell ratio. The seeded cells were treated with various anti-human IgG conjugates, including 0.5 μg/mL of an anti-CD3 IgG (Clone 1) conjugated to oligonucleotide A; 0.5 μg/mL of an anti-CD3 IgG (clone 2) conjugated to oligonucleotide E; and 0.5 μg/mL of an anti-TCRαβ IgG (Clone 1) conjugated to oligonucleotide F. All treatment conditions also included co-stimulation with 1 μg/mL anti-human CD28 IgG (clone CD28.2) for the duration of the 72-hour incubation period. Flow cytometric analysis of CD25 activation marker expression on both CD4+ and CD8+ T cell populations reveal mediated stimulatory responses for oligo conjugates binding to various components of the T cell receptor.

### Example 5: Quenching Stimulation of Target Cells by Reacting Cell Stimulation Compositions with a Complementary Oligo

The ability of the compositions made in accordance with Example 1 (unless commercially purchased pre-conjugated) to stimulate and quench a response in the enriched cells of Example 2 was tested.

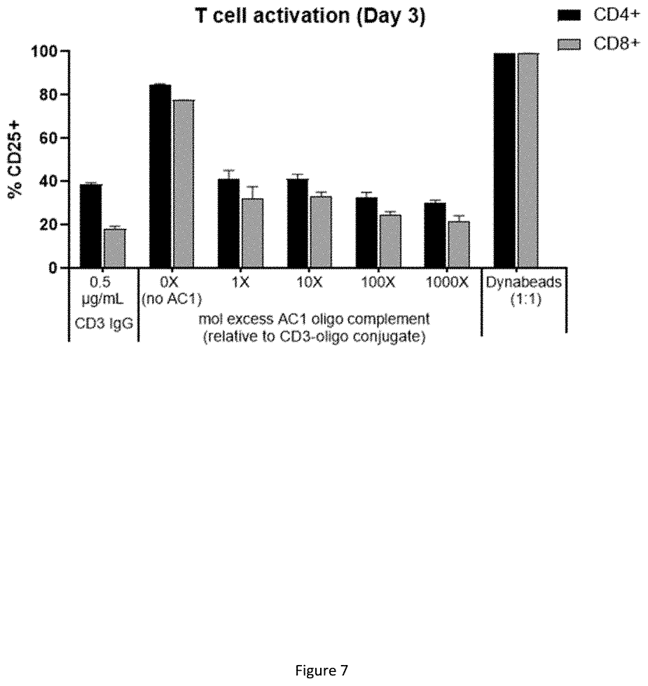

For human T cells, it is shown in FIG. 7 that stimulation of cells using compositions of this disclosure may be quenched using nucleic acid polymers complementary to at least a portion of the nucleic acid polymer conjugated to the binding moiety. Briefly, 1.6×10⁵ cells were seeded per well of a 96-well plate and contacted with a cell stimulation composition and titrated concentrations of a complementary nucleic acid polymer. Control condition included treatment of the seeded cells with 0.5 μg/mL unmodified anti-human CD3 IgG (clone 1) along with 1 μg/mL CD28 IgG (clone CD28.2) as well as Dynabeads™ incubated at a 1:1 bead per cell ratio. Seeded cells were treated with various preparations of anti-human CD3 IgG (clone 1) conjugated to oligonucleotide A (0.5 μg/mL) pre-incubated with a 1×, 10×, 100× or 1000× concentration of a complementary oligonucleotide (AC1). All treatments included co-stimulation with 1 μg/mL anti-human CD28 IgG (clone CD28.2). Following the 72-hour incubation period, cells were analyzed for CD25 activation marker expression. Flow cytometry analysis of stimulated T cells reveal a dose-dependent modulation of activation when conjugates are pre-incubated with increasing concentrations of an nucleic acid polymer complementary to at least a portion of the nucleic acid polymer conjugated to the binding moiety of the cell stimulation composition.

### Example 6: Modulating Stimulation of Target Cells by Timing the Hybridization of Cell Stimulation Compositions with a Complementary Oligo

The stimulation of cells enriched as in Example 2 using the compositions made in accordance with Example 1 (unless commercially purchased pre-conjugated) could be modulated using nucleic acid polymers complementary to at least a portion of the nucleic acid polymer conjugated to the binding moiety.

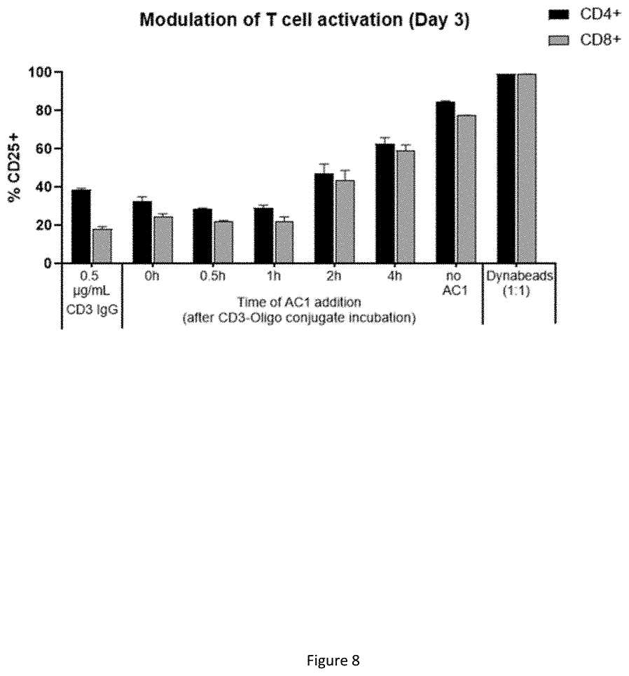

For human T cells, FIG. 8 demonstrates the possibility of modulating the duration of target cells stimulation by timing the addition of a nucleic acid polymer complementary to at least a portion of the nucleic acid polymer conjugated to the binding moiety. Briefly, 1.6×10⁵ cells were seeded per well of a 96-well plate and contacted with a cell stimulation composition. Following the initial stimulation, the cell stimulation composition was contacted with a complementary oligonucleotide (AC1) at different time intervals and allowed to proceed with incubation for 72-hours at 37° C. Control conditions included the treatment of seeded cells with unmodified anti-human CD3 IgG (0.5 μg/mL; clone 1) along with anti-CD28 IgG (1.0 μg/mL; clone CD28.2) as well as treatment with Dynabeads™ incubated at a 1:1 bead per cell ratio. All treatment wells were contacted with anti-human CD3 IgG (clone 1) conjugated to oligonucleotide A in the presence of anti-CD28 IgG (1 μg/mL; clone CD28.2). Modulation of activation was achieved by adding a 300× concentration of the complementary nucleic acid polymer (oligonucleotide AC1) to the treated wells at 0, 0.5, 1, 2 and 4 hours following initial stimulation with CD28 and CD3-oligo conjugates, and left to incubate for 72 hours at 37° C. A treatment condition that omitted the addition of the complementary oligo (no AC1 condition) was also included as a benchmark of maximum activation potential for the experiment. Flow cytometry analysis for CD25 marker expression reveals that stimulation of the target cells using the cell stimulation composition could be modulated in a temporally-dependent manner; decreased overall stimulation was measured the earlier that the cell stimulation composition was exposed to the nucleic acid polymer complementary to at least a portion of the nucleic acid polymer conjugated to the binding moiety of the cell stimulation composition.

### Example 7: Modifying the Stimulation Potential of Cell Stimulation Compositions with Point Mutations

Improved or worsened stimulation of target cells (enriched as in Example 2) was tested by engineering point mutations to modify melting temperature of predicted secondary structures into the nucleic acid polymers of cell stimulation compositions made in accordance with Example 1 (unless commercially purchased pre-conjugated).

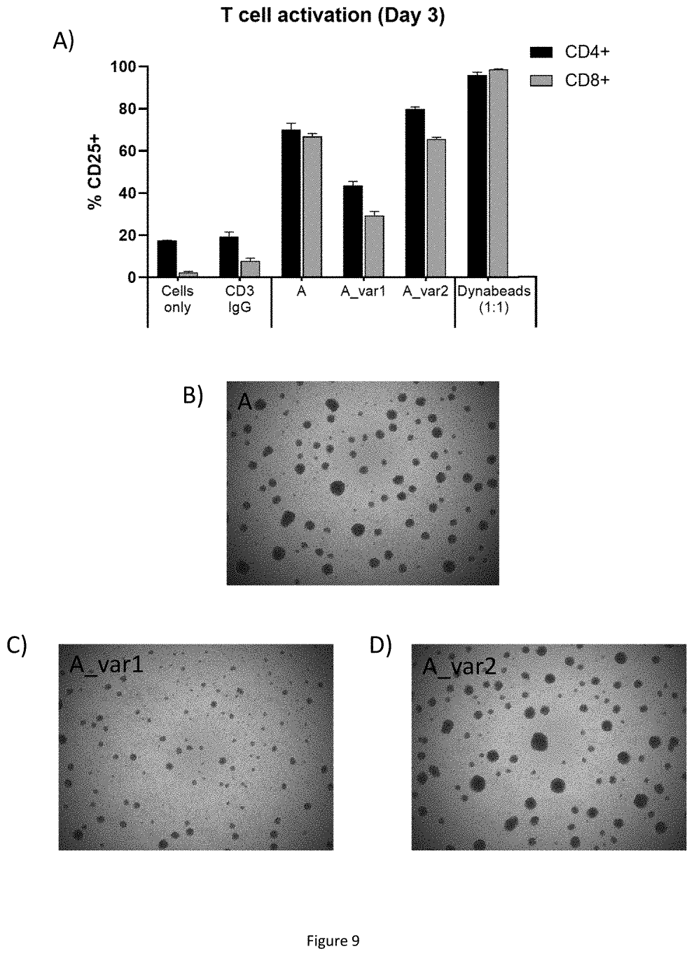

For human T cells, it is shown in FIGS. 9 to 11 that the stimulation potential of cell stimulation compositions could be modified via engineered point mutations in component nucleic acid polymers. Briefly, 1.6×10⁵ cells were seeded per well of a 96-well plate and contacted with various compositions to test for activation (measured via CD25 marker expression) following a 72-hour incubation period at 37° C. All treatments included co-stimulation with 1 μg/mL anti-human CD28 IgG (cloneCD28.2) for the duration of the 72-hour incubation period.

In FIG. 9A, a first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). A third control condition also included Dynabeads™ incubated at a 1:1 bead per cell ratio. The seeded cells were contacted with 0.5 μg/mL of anti-human CD3 IgG (clone 1) conjugated to: oligonucleotide A (having a predicted melting temperature of predicted secondary structures of ^{˜}38° C.); 0.5 μg/mL of anti-human CD3 IgG (clone 1) conjugated with oligonucleotide A_var1 (having a predicted melting temperature of predicted secondary structures of ^{˜}52° C.); and 0.5 μg/mL of anti-human CD3 IgG (clone 1) conjugated with oligonucleotide A_var2 (having a predicted melting temperature of predicted secondary structures of ^{˜}31° C.). The cell stimulation composition including oligonucleotide A_var1 resulted in significantly reduced CD25 marker expression in both CD4⁺ and CD8⁺ T cell populations, compared to a cell stimulation composition that included oligonucleotide either A or A_var2. In addition, reduced T cell clustering is observed for the cell stimulation composition including oligonucleotide A_var1 (FIG. 9C), compared to those observed for A (FIG. 9B) or A_var2 (FIG. 9D). Collectively, the results suggest an enhanced output of T cell activation as melting temperature of predicted secondary structures is decreased.

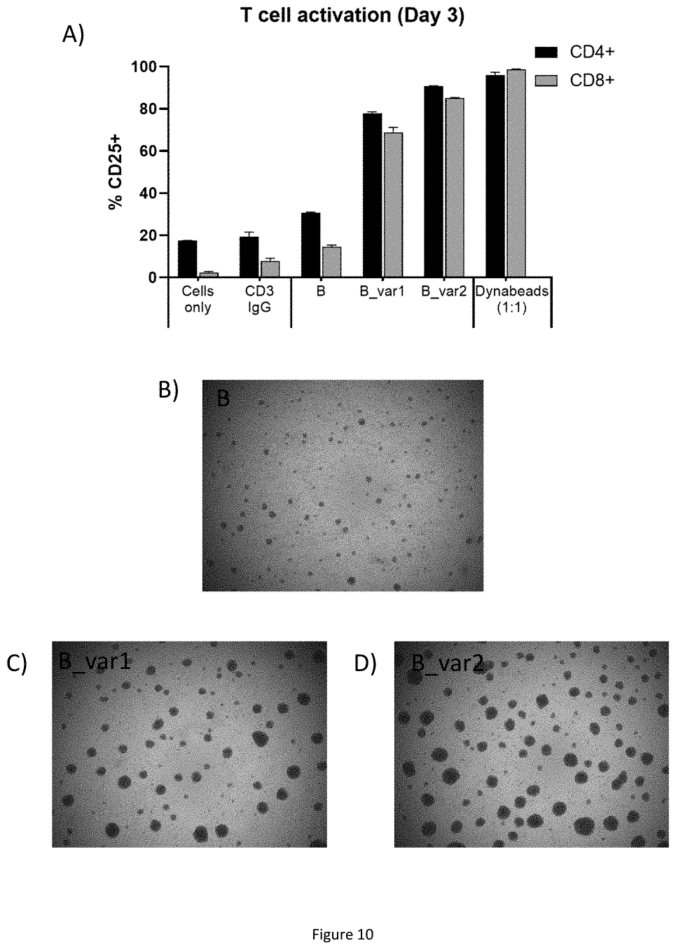

The results obtained from the previous set of experimentation with oligonucleotide variants A were expanded to include additional nucleic acid polymer variant of a different identity. FIG. 10 demonstrates a similar trend of enhanced output of activation when strategically placed point mutations in the nucleic acid polymer sequences of B_var1 are included to reduce the melting temperature of predicted secondary structures from about 45° C. to about 28° C. in B_var2. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). A third control condition also included Dynabeads™ incubated at a 1:1 bead per cell ratio. The seeded cells were contacted with 0.5 μg/mL of anti-human CD3 IgG (clone 1) conjugated either to oligonucleotide B (having a predicted melting temperature of predicted secondary structures of known nucleotides of ^{˜}45° C.); 0.5 μg/mL of anti-human CD3 IgG (clone 1) conjugated with oligonucleotide B_var1 (having a predicted melting temperature of predicted secondary structures of ^{˜}44° C.); or B_var2 (having a predicted melting temperature of predicted secondary structures of ^{˜}28° C.). Flow cytometry analysis of CD25 marker expression after 72 hrs incubation at 37° C. show enhanced activation levels in both CD4⁺ and CD8⁺ T cell populations for the cell stimulation composition that included oligonucleotide B_var2 relative to the cell stimulation composition that included oligonucleotide B_var1 or B. Likewise, enhanced T cell clustering is also observed for the cell stimulation composition including oligonucleotide B_var2 (FIG. 10D), compared to those observed for B (FIG. 10B) or B_var1 (FIG. 10C)

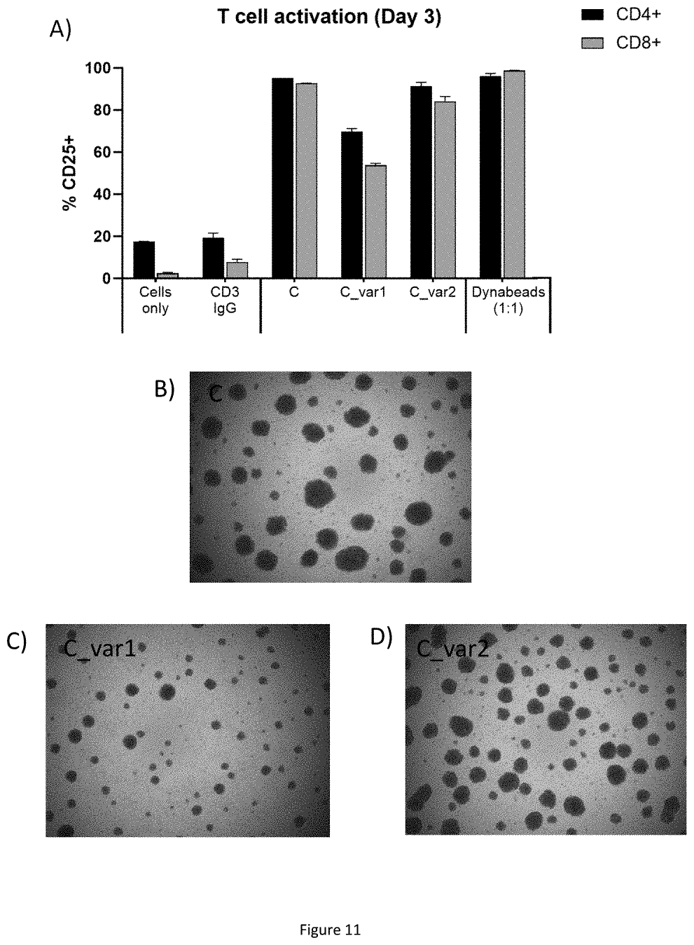

A third nucleic acid polymer variant set was tested in FIG. 11. Strategically placed point mutations in the nucleic acid polymer sequences of C_var1 were included to reduce the melting temperature of predicted secondary structures from about 49° C. to about 34° C. in C_var2. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). A third control condition also included Dynabeads™ incubated at a 1:1 bead per cell ratio. The seeded cells were contacted with 0.5 μg/mL of anti-human CD3 IgG (clone 1) conjugated either to oligonucleotide C_var1, C_var2 or C (having a predicted melting temperature of predicted secondary structures of known nucleotides of ^{˜}29° C.). Flow cytometry analysis of CD25 marker expression after 72 hrs incubation at 37° C. show reduced activation levels in both CD4′ and CD8′ T cell populations for the cell stimulation composition that included oligonucleotide C_var1 relative to the cell stimulation composition that included either oligonucleotide C or C_var2. Likewise, reduced T cell clustering is also observed for the cell stimulation composition that includes oligonucleotide C_var1 (FIG. 11C), compared to those observed for C (FIG. 11B) or C_var2 (FIG. 11D).

### Example 8: Quenching Stimulation of Target Cells by Reacting Cell Stimulation Compositions with Various Complementary Oligonucleotides

The ability of the compositions made in accordance with Example 1 (unless commercially purchased pre-conjugated) to stimulate and quench a response in the enriched cells of Example 2 was tested.

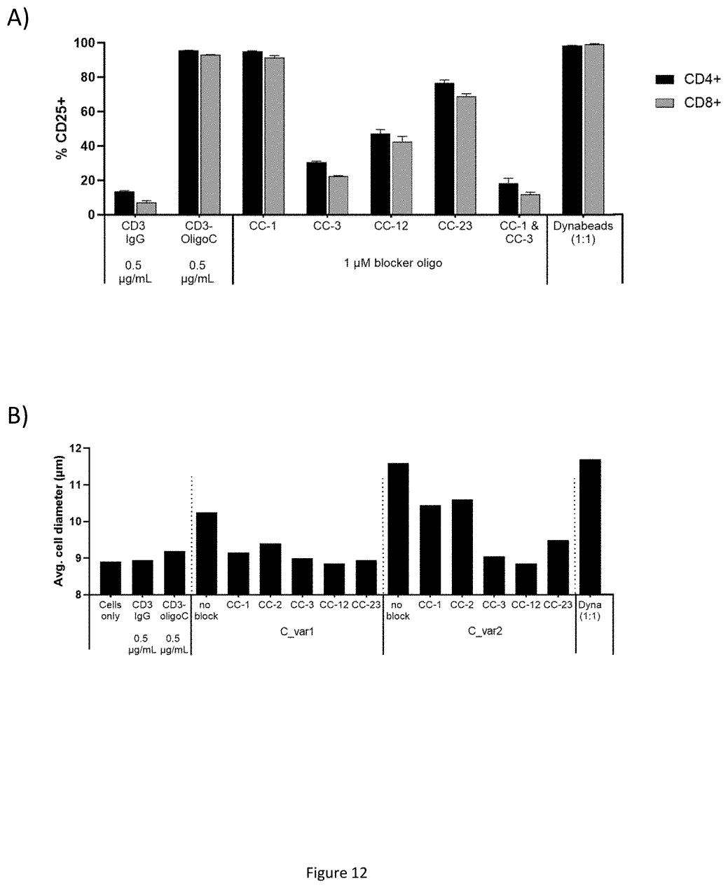

For human T cells, it is shown in FIG. 12 that stimulation of cells using compositions of this disclosure may be quenched using various nucleic acid polymers having complementarity along the different portions of the length of the nucleic acid polymer conjugated to the binding moiety. Briefly, 1.6×10⁵ cells were seeded per well of a 96-well plate and contacted with a cell stimulation composition and a complementary nucleic acid polymer. Control condition included treatment of the seeded cells with 0.5 μg/mL unmodified anti-human CD3 IgG (clone 1) along with 1 μg/mL CD28 IgG (clone CD28.2) as well as Dynabeads™ incubated at a 1:1 bead per cell ratio. Seeded cells were treated with anti-human CD3 IgG (clone 1) conjugated to oligonucleotide C (0.5 μg/mL) pre-incubated with 1 μM of either complementary oligonucleotide CC-1, CC-3, CC-12, CC-23, or both CC-1 and CC-3. All treatments included co-stimulation with 1 μg/mL anti-human CD28 IgG (clone CD28.2). Following the 72-hour incubation period, cells were analyzed for CD25 activation marker expression (FIG. 12A). Flow cytometry analysis of the stimulated T cells revealed that CC-3 alone had the most profound effect on quenching T cell activation, while CC-1 alone did not appear to have an effect on quenching T cell activation.

Responsiveness of C_var1 and C_var 2 (conjugated with anti-human CD3 IgG) to 1 μM of either complementary oligonucleotide CC-1, CC-3, CC-12, CC-23, or both CC-1 and CC-3 was also assessed. As above, 1.6×10⁵ cells were seeded per well of a 96-well plate and contacted with a cell stimulation composition and a complementary nucleic acid polymer. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). A third control condition also included Dynabeads™ incubated at a 1:1 bead per cell ratio. Seeded cells were treated with various preparations of anti-human CD3 IgG (clone 1) conjugated to oligonucleotide C_var1 or C_var2 (0.5 μg/mL) pre-incubated with 1 μM of either complementary oligonucleotide CC-1, CC-3, CC-12, CC-23, or both CC-1 and CC-3. All treatments included co-stimulation with 1 μg/mL anti-human CD28 IgG (clone CD28.2). Following the 72-hour incubation period, cells for each individual condition were resuspended and analyzed for their diameter, the average of the population diameter reported in FIG. 12B. Reduced diameter among the conditions including complementary oligonucleotide pre-hybridization suggests reduced activation of T cells in comparison to T cells activated using antibody conjugated C_var1 or C_var2 (no block).

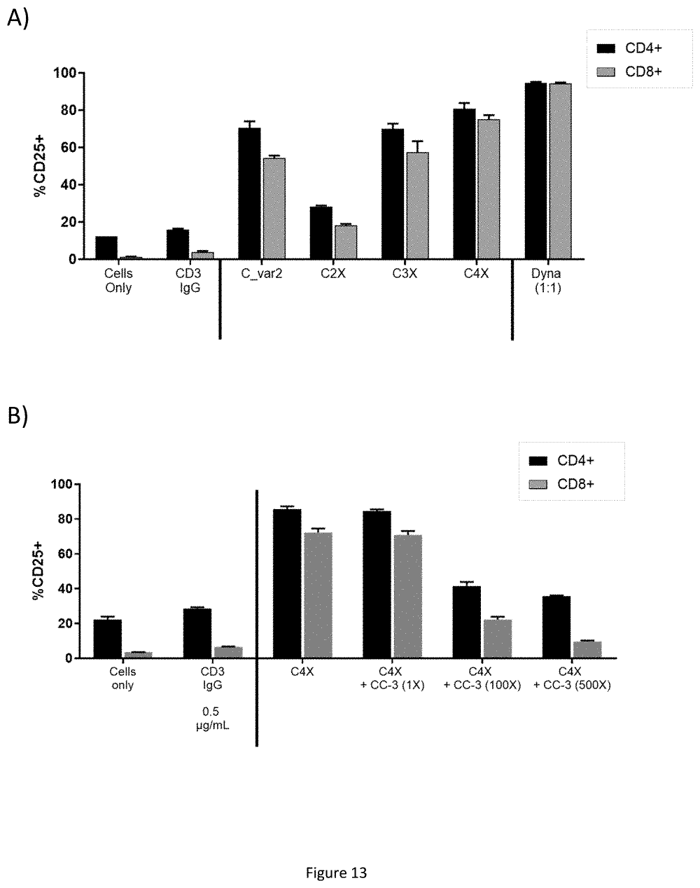

It was observed in FIG. 12A that the most distal portion (complementary to oligonucleotide CC-3) of the oligonucleotide conjugated to the anti-human CD3 antibody was the most susceptible to quench cell activation. Accordingly, further oligonucleotides based on the sequence complementary to oligonucleotide CC-3 were designed and tested in T cell activation experiments (FIG. 13A). Briefly, 2, 3 or 4 units of the foregoing sequence were concatenated, being sure that in the concatenation, the subsequent unit was reversed relative to the preceding unit (e.g. CCCATATAAGAAATTTAAAGAATATACCC) in order to maintain a favorable (low) melting temperature of secondary structures. The resulting concatenations were termed C2×, C3× or C4×, and were tested in comparison C_var2 (each conjugated to anti-human CD3 IgG). As above, 1.6×10⁵ cells were seeded per well of a 96-well plate and contacted with a cell stimulation composition. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). A third control condition also included Dynabeads™ incubated at a 1:1 bead per cell ratio. Seeded cells were treated with anti-human CD3 IgG (clone 1) conjugated to oligonucleotide C_var2, C2×, C3×, or C4× (each 0.5 μg/mL). All treatments included co-stimulation with 1 μg/mL anti-human CD28 IgG (clone CD28.2). In this instance, a 48-hour incubation period was chosen in order to capture the differences in activation stimulus for each oligonucleotide variants. Following the 48-hour incubation period, cells were analyzed for CD25 expression (FIG. 13A). Increased activation of T cells was observed as the number of concatenated units increased.

Since C4× (conjugated to anti-human CD3 IgG) resulted in the highest activation of T cells, this cell stimulation composition was tested in quenching experiments essentially as described above. Briefly, 1.6×10⁵ cells were seeded per well of a 96-well plate and contacted with a cell stimulation composition and titrated concentrations of a complementary nucleic acid polymer. A first control condition left the seeded cells untreated (cells only), while a second control condition contacted the seeded cells with 0.5 μg/mL unmodified anti-human CD3 IgG (clone 1) and 1 μg/mL anti-human CD28 IgG (clone CD28.2). Seeded cells were treated with various preparations of anti-human CD3 IgG (clone 1) conjugated to oligonucleotide C4× (0.5 μg/mL) pre-incubated with a 1×, 100×, and 500× relative concentration of a complementary oligonucleotide (CC-3). All treatments included co-stimulation with 1 μg/mL anti-human CD28 IgG (clone CD28.2). Following a 72-hour incubation period, cells were analyzed for CD25 activation marker expression (FIG. 13B). Flow cytometry analysis of stimulated T cells revealed a dose-dependent modulation of activation when conjugates are pre-incubated with increasing concentrations of a nucleic acid polymer complementary to at least a portion of the nucleic acid polymer conjugated to the binding moiety of the cell stimulation composition.

### Example 9: Stimulating Target Cells with Bispecific Complexes of Binding Moieties

The ability of the compositions made in accordance with Example 1 (unless commercially purchased pre-conjugated) to stimulate a response in the enriched cells of Example 2 was tested.

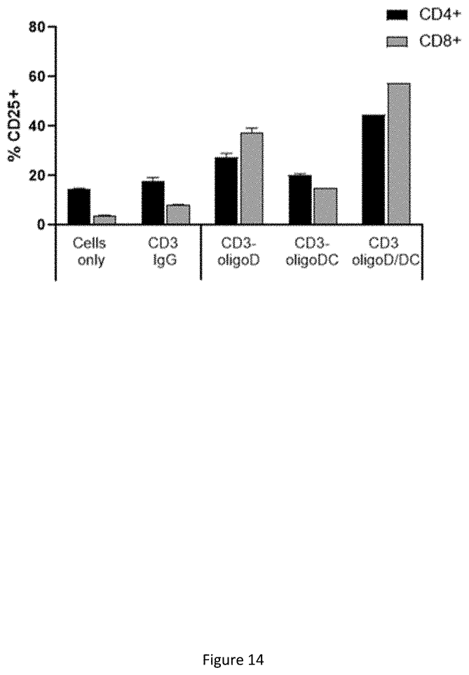

For human T cells, it is shown in FIG. 14 that compositions of this disclosure are capable of stimulating T cells. Here, the compositions comprise a first anti-human anti-CD3 IgG (clone 1) antibody conjugated to oligonucleotide D and a second anti-human anti-CD3 IgG (clone 1) antibody conjugated to oligonucleotide DC, largely complementary to oligonucleotide D. Five micrograms of each anti-CD3-oligo conjugate were combined in a vial and incubated at room temperature for 15 minutes to allow hybridization of conjugates to occur, thereby forming bispecific complexes of binding moieties. Briefly, 1.6×10⁵ cells were seeded per well of a 96-well plate and contacted with a cell stimulation composition. Control condition included unstimulated cells and treatment of the seeded cells with 0.5 μg/mL unmodified anti-human CD3 IgG (clone 1) along with 1 μg/mL CD28 IgG (clone CD28.2). Seeded cells were treated with individual anti-human CD3 IgG (clone 1) conjugates (0.5 μg/mL of either oligonucleotide D or DC conjugate) or 0.5 μg/mL of the previously hybridized conjugates. All treatments included co-stimulation with 1 μg/mL anti-human CD28 IgG (clone CD28.2). Following a 72-hour incubation period, cells were analyzed for CD25 activation marker expression by flow cytometry. Individual anti-CD3 conjugates resulted in partial T cell activation that was further enhanced upon complexing a portion of the nucleic acid polymer conjugated to the binding moiety of the cell stimulation composition.

### Example 10: Stimulating Isolated Target Cells with Cell Stimulation Compositions and Costimulatory Molecules

The ability of the compositions made in accordance with Example 1 (unless commercially purchased pre-conjugated) to stimulate a response among T cells isolated from a mouse spleen sample. Briefly, the spleens were harvested from C57Bl/6J mice in accordance with animal welfare guidelines, and the spleens were disrupted in PBS containing 2% fetal bovine serum (FBS). Aggregates and debris were removed by passing the cell suspension through a 70 μm mesh nylon strainer. The collected cells were centrifuged at 300×g for 10 minutes and resuspended at 1.0×10⁸ nucleated cells/mL before T cell isolation using the EasySep Mouse T cell isolation kit (STEMCELL Technologies Inc., cat no 19851) according to the manufacturer's instructions.

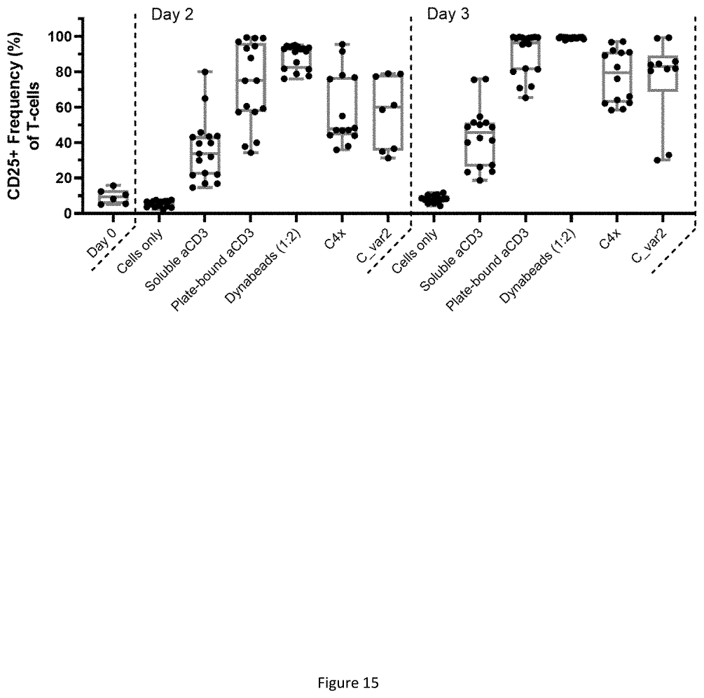

For mouse T cells, it is shown in FIG. 15 that compositions of this disclosure are capable of stimulating T cells. Here, the composition comprises anti-mouse anti-CD3 IgG (clone 4) antibody conjugated to oligonucleotide C4× or to oligonucleotide C_var2. Briefly, 2.0×10⁵ cells were seeded per well of a 96-well plate and contacted with a cell stimulation composition. A first control condition left the seeded cells untreated (cells only), while a second control condition contacted the seeded cells with 0.5 μg/mL unmodified anti-mouse CD3 IgG (clone 4) and 2 μg/mL anti-mouse CD28 IgG. A third control condition also included Dynabeads™ incubated at a 1:2 bead per cell ratio. A fourth condition tested plate-bound anti-mouse CD3 IgG (clone 4). Seeded cells were treated with 0.5 μg/mL anti-mouse CD3 IgG (clone 4) conjugated to either oligonucleotide C4× or to oligonucleotide C_var2. All treatments included co-stimulation with 2 μg/mL anti-mouse CD28 IgG. Following a 48-hour or a 72-hour incubation period, cells were analyzed for CD25 activation marker expression by flow cytometry. Individual soluble anti-CD3 conjugates resulted in partial T cell activation that could be enhanced when the anti-CD3 antibodies were conjugated to either of the tested oligonucleotides. The cell stimulation compositions could achieve cell activation in comparable efficiencies to the Dynabeads condition and the plate-bound anti-CD3 antibody condition.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A cell stimulation composition, comprising:
a first binding moiety capable of binding a target antigen; and
a first nucleic acid polymer conjugated directly or indirectly to the first binding moiety, a sequence of the first nucleic acid polymer comprising a melting temperature of predicted secondary structure(s) about 60° C. or less.

2. The composition of claim 1, wherein the first binding moiety is an antibody, or a fragment thereof, a peptide, an aptamer, an affimer, or a small molecule.

3. The composition of claim 1, wherein the target antigen is a component of the T cell receptor complex, and the component of the T cell receptor complex is CD3 epsilon, CD3 delta, CD3 gamma, CD3 zeta, TCR alpha, TCR beta, TCR gamma, or TCR delta.

4. (canceled)

5. The composition of claim 1, wherein a predicted free energy of the first nucleic acid polymer to self-hybridize at 150 mM monovalent ion concentration is between about +1 kcal/mol and −10 kcal/mol.

6. The composition of claim 5, wherein the predicted free energy of the first nucleic acid polymer at 150 mM monovalent ion concentration is negative.

7. The composition of claim 1, wherein the melting temperature of predicted secondary structure(s) of the first nucleic acid polymer is between about 50° C. and 20° C. or less.

8. The composition of claim 1, wherein the first nucleic acid polymer is at least 10 nucleotides.

9. The composition of claim 1, wherein the first nucleic acid polymer or a complementarity region thereof is complementary to a second nucleic acid polymer.

10. The composition of claim 9, wherein the second nucleic acid polymer is directly or indirectly conjugated to a second binding moiety.

11. The composition of claim 10, wherein a bispecific complex of binding moieties is formed by hybridization of the first nucleic acid polymer and the second nucleic acid polymer.

12. The composition of claim 11, wherein the first binding moiety and the second binding moiety bind the same target antigen or different target antigens.

13-17. (canceled)

18. A method of stimulating target cells in a sample of cells, the method comprising:
a) contacting the sample of cells with a first binding moiety conjugated directly or indirectly to a first nucleic acid polymer, the first binding moiety capable of binding a target antigen on the target cells, wherein the first binding moiety conjugated directly or indirectly to a first nucleic acid polymer forms a cell stimulation composition;
b) incubating the target cells having been bound by the first binding moiety of the cell stimulation composition; and
c) optionally, contacting the sample of cells with a co-stimulatory binding moiety capable of binding a co-stimulatory antigen on the target cells, either before, contemporaneous with the cell stimulation composition, or after contacting the sample of cells with the cell stimulation composition.

19. The method of claim 18, wherein the first binding moiety is an antibody, or a fragment thereof, a peptide, an aptamer, an affimer, or a small molecule.

20. The method of claim 18, wherein a predicted free energy of the first nucleic acid polymer to self-hybridize at 150 mM monovalent ion concentration is between about +1 kcal/mol and −10 kcal/mol.

21. The method of claim 20, wherein the predicted free energy of the first nucleic acid polymer at 150 mM monovalent ion concentration is negative.

22. The method of claim 18, wherein the first nucleic acid polymer comprises a melting temperature of predicted secondary structures about 60° C. or less, and preferably between about 50° C. and 20° C. or less.

23. (canceled)

24. The method of claim 18, wherein the first nucleic acid polymer is at least 10 nucleotides.

25. The method of claim 18 wherein the target cells are T cells, NK cells, or B cells.

26. The method of claim 25, wherein the first target antigen:
i. for T cells is CD3 epsilon, CD3 delta, CD3 gamma, CD3 zeta, TCR alpha, TCR beta, TCR gamma, or TCR delta;
ii. for NK cells is NKp30, NKp44, and NKp46, a C-type lectin-like receptor such as NKG2D, CD335, CD94, CD2, CD16 binding to Fc regions of antibodies, CD122, CD132, IL-15 receptor alpha; and
iii. for B cells is CD40, an anti-immunoglobulin, CD79a, or CD79b.

27. The method of claim 26, further comprising:
d) increasing proliferation of the target cells after step c).

28-29. (canceled)

30. The method of claim 18, further comprising hybridizing a second nucleic acid polymer to the first nucleic acid polymer or a complementarity region thereof.

31. The method of claim 30, wherein a complementarity of the second nucleic acid polymer to the first nucleic acid polymer, or the complementary region thereof, is between about 40% and 100%.

32. The method of claim 30, wherein hybridizing the second nucleic acid polymer to the first nucleic acid polymer, or the complementarity region thereof, before, contemporaneous with, or after contacting the sample of cells with the second binding moiety quenches or modulates a level of target cell stimulation.

33. The method of claim 32, further comprising displacing the second nucleic acid polymer from the first nucleic acid polymer, or the complementarity region thereof, by competition with a competitor nucleic acid polymer, the competitor nucleic acid polymer having a higher degree of complementarity for the second nucleic acid polymer than the second nucleic acid polymer for the first nucleic acid polymer or the complementarity region thereof.

34. The method of claim 30, wherein the second nucleic acid polymer is directly or indirectly conjugated to a second binding moiety.

35-50. (canceled)


SEQUENCE LISTING (21 sequences)

SEQ ID NO: 1 - DNA, 69 bp, Artificial Sequence
  Synthetic construct
  misc_feature (37)..(37): n is a, c, g, or t
ccttggcacccgagaattccactcattgtaactcctnaaaaaaaaaaaaaaaaaaaaaaaaaaaaaaaa

SEQ ID NO: 2 - DNA, 36 bp, Artificial Sequence
  Synthetic construct
aggagttacaatgagtggaattctcgggtgccaagg

SEQ ID NO: 3 - DNA, 40 bp, Artificial Sequence
  Synthetic construct
ccttggcacccgagaattccatatcccttgggatggaaaa

SEQ ID NO: 4 - DNA, 40 bp, Artificial Sequence
  Synthetic construct
cctttgcacccgagaattccatatcccttgtgatagaaaa

SEQ ID NO: 5 - DNA, 90 bp, Artificial Sequence
  Synthetic construct
  misc_feature (35)..(44): n is a, c, g, or t
  misc_feature (60)..(68): n is a, c, g, or t
gtgactggagttcagacgtgtgctcttccgatctnnnnnnnnnnctcattgtaactcctnnnnnnnnngctttaaggccggtcctagcaa

SEQ ID NO: 6 - DNA, 70 bp, Artificial Sequence
  Synthetic construct
gtgactggagttcagacgtgtgctcttccgatcctcattgtaactcctgctttaaggccggtcctagcaa

SEQ ID NO: 7 - DNA, 71 bp, Artificial Sequence
  Synthetic construct
gtgattgtagttcagatgtgtgctcttccgatctctcattgtaactccttctttaagtccggtcctagcaa

SEQ ID NO: 8 - DNA, 69 bp, Artificial Sequence
  Synthetic construct
  misc_feature (23)..(32): n is a, c, g, or t
  misc_feature (48)..(56): n is a, c, g, or t
cggagatgtgtataagagacagnnnnnnnnnnctcattgtaactcctnnnnnnnnncccatataagaaa

SEQ ID NO: 9 - DNA, 50 bp, Artificial Sequence
  Synthetic construct
cggagatgtgtataagagacagctcattgtaactcctcccatataagaaa

SEQ ID NO: 10 - DNA, 69 bp, Artificial Sequence
  Synthetic construct
cggagatgtgtataagagacagttttttttttctcattgtaactccttttttttttcccatataagaaa

SEQ ID NO: 11 - DNA, 35 bp, Artificial Sequence
  Synthetic construct
ggcttttacttctatctctttctttaacactttct

SEQ ID NO: 12 - DNA, 44 bp, Artificial Sequence
  Synthetic construct
gaaagtgttaaagaaagagatagaagtaaaagcctcgatacgta

SEQ ID NO: 13 - DNA, 69 bp, Artificial Sequence
  Synthetic construct
  misc_feature (37)..(37): n is a, c, g, or t
ccttggcacccgagaattccatatcccttgggatggnaaaaaaaaaaaaaaaaaaaaaaaaaaaaaaaa

SEQ ID NO: 14 - DNA, 69 bp, Artificial Sequence
  Synthetic construct
  misc_feature (37)..(37): n is a, c, g, or t
ccttggcacccgagaattccacgtaacgtagagcganaaaaaaaaaaaaaaaaaaaaaaaaaaaaaaaa

SEQ ID NO: 15 - DNA, 22 bp, Artificial Sequence
  Synthetic construct
ctgtctcttatacacatctccg

SEQ ID NO: 16 - DNA, 40 bp, Artificial Sequence
  Synthetic construct
aggagttacaatgagtttctgtctcttatacacatctccg

SEQ ID NO: 17 - DNA, 13 bp, Artificial Sequence
  Synthetic construct
tttcttatatggg

SEQ ID NO: 18 - DNA, 31 bp, Artificial Sequence
  Synthetic construct
tttcttatatgggtttaggagttacaatgag

SEQ ID NO: 19 - DNA, 29 bp, Artificial Sequence
  Synthetic construct
cccatataagaaatttaaagaatataccc

SEQ ID NO: 20 - DNA, 45 bp, Artificial Sequence
  Synthetic construct
cccatataagaaatttaaagaatataccctttcccatataagaaa

SEQ ID NO: 21 - DNA, 61 bp, Artificial Sequence
  Synthetic construct
cccatataagaaatttaaagaatataccctttcccatataagaaatttaaagaatataccc